(19) United States
(12) Patent Application Publication
(10) Pub. No.: US 2005/0287655 A1
(43) Pub. Date: Dec. 29, 2005

(54) Process for producing mevalonic acid

(75) Inventors: Kazuhiko Tabata, Tokyo (JP); Shin-chi Hashimoto, Tokyo (JP)

Correspondence Address: FITZPATRICK CELLA HARPER & SCINTO, 30 ROCKEFELLER PLAZA, NEW YORK, NY, 10112, US

(73) Assignee: Kyowa Hakko Kogyo Co., Ltd., Tokyo (JP)

(21) Appl. No.: 10/513,759

(22) PCT Filed: May 8, 2003
(86) PCT No.: PCT/JP03/05765

§ 371 Date: Apr. 8, 2005

(30) Foreign Application Priority Data
May 10, 2002 (JP) 2002-135073

(51) Int. Cl. C12P 7/40; C12N 1/21

(52) U.S. Cl. 435/136; 435/252.3

(57) ABSTRACT

A microorganism having the ability to biosynthesize mevalonic acid from acetyl-CoA is obtained by transfecting a DNA participating in biosynthesis of mevalonic acid from acetyl-CoA into a host microorganism, preferably into a microorganism having only a non-mevalonic acid pathway, to express the DNA therein. Mevalonic acid can be efficiently produced by culturing the microorganism, and recovering mevalonic acid that is produced and accumulated in a large amount in the culture.

### TECHNICAL FIELD

The present invention relates to a recombinant microorganism which is provided an ability to produce mevalonic acid, and a process for producing mevalonic acid using the microorganism.

### BACKGROUND ART

Mevalonic acid is a substance isolated first by Wright et al., [J. Am. Chem. Soc., 78, 5273, 1956]. In an organism, mevalonic acid has been known to be an intermediate in biosynthesis of isopentenyl pyrophosphate, which provides a basis of the structure of various types of isoprenoid compounds that include cholesterol [Harper's review of Biochemistry (19th edition), Lange Medical Publications (1983)]. Isopentenyl pyrophosphate biosynthetic pathway via mevalonic ac id is referred to as a mevalonic acid pathway, which is as follows. Acetyl coenzyme A (hereinafter, abbreviated as acetyl-CoA), which is generated by glycolysis and the like, is converted to acetoacetyl-CoA by the activity of acetyl-CoA acetyltransferase, and further converted to 3-hydroxy-3-methylglutaryl coenzyme A (hereinafter, abbreviated as HMG-CoA) by the activity of HMG-CoA synthase, followed by production of mevalonic acid by the activity of HMG-CoA reductase. Mevalonic acid is converted to mevalonate-5-phosphate by the activity of mevalbnate kinase, and then converted to mevalbnate-5-diphosphate by the activity of phosphomevalonate kinase. Finally, isopentenyl pyrophosphate is produced by the activity of diphosphomevalonate decarboxylase.

Although mevalonic acid exhibits two types of structures, an acid form and a lactone form, it has been known that they can be converted each other.

Mevalonic acid is used as a growth promoter for a variety of microorganisms, plants and the like, and is used as synthetic precursors of pyrethroid pesticides, ubiquinone (respiratory coenzyme), dolichol (essential factor for biosynthesis of polysaccharides), lipid-soluble vitamin and the like. Furthermore, effects of mevalonic acid on the skin, such as the ability to impart adequate moisturized feel and softness (Japanese Published Unexamined Patent Application No. 099707/1992) and an anti-aging action (Japanese Published Unexamined Patent Application No. 338626/1998), were reported in recent years, and it is used as an ingredient of cosmetics. Conventionally, a chemically synthesized racemic form has been used in these researches, and it has been difficult to obtain naturally occurring mevalonic acid [R(+) form that is present in an organism].

Although production of naturally occurring mevalonic acid by a fermentation method was reported as an example in which Saccharomycopsis fibuligera NRRL Y-7069 is used [U.S. Pat. No. 3,617,447; Appl. Microbiol., 16, 965 (1968)), the yield thereby is low, which may be just 700 to 1000 μg/mL, not leading to industrial production. Further, productivity is improved to approximately 20 g/L using Saccharomycopsis fibuligera IF00107 [J. Ferm. Bioeng., 86, 58 (1989)], ADK8107, ADK8108 (Japanese Published Unexamined Patent Application No. 210174/1991) that belong to the same species, more recently. However a cultivation time is as long as 200 hrs, and the yield from the sugar is low. Accordingly, it is far from an industrially useful process for the production.

### DISCLOSURE OF THE INVENTION

A efficient process for producing mevalonic acid that can be practiced in industrial production has been desired.

In biosynthesis of isopentenyl pyrophosphate, a biosynthetic pathway not via mevalonic acid (non-mevalonic acid pathway) has been found in microorganisms and plants [Protein Nucleic acid and Enzyme, 42, 2590 (1997)], in addition to a biosynthetic pathway via mevalonic acid (mevalonic acid pathway). The present inventors elaborately carried out the investigation on the basis of a hypothesis that a microorganism having only a non-mevalonic acid pathway but not having a mevalonic acid pathway does not metabolize mevalonic acid, thereby enabling extremely efficient production of mevalonic acid, and thus, the present invention was accomplished.

The present invention provides the following items (1) to (32).

(1) A process for producing mevalonic acid, which comprises:

culturing a microorganism having the ability to biosynthesize mevalonic acid from acetyl coenzyme A (acetyl-CoA) in a medium, the microorganism being obtainable by transfecting a DNA participating in biosynthesis of mevalonic acid from acetyl-CoA into a host microorganism;

allowing production and accumulation of mevalonic acid in the culture; and

recovering mevalonic acid from the culture.

(2) The process according to the above item (1), wherein the host microorganism is a microorganism not having the ability to biosynthesize mevalonic acid from acetyl-CoA.

(3) The process according to the above item (2), wherein the microorganism not having the ability to biosynthesize mevalonic acid from acetyl-CoA is a microorganism not having a mevalonic acid pathway.

(4) The process according to-any one of the above items (1) to (3), wherein the DNA to be transfected is one or more DNA(s) selected from the group consisting of a DNA encoding an enzyme having the activity to produce acetoacetyl-CoA from acetyl-CoA, a DNA encoding an enzyme having the activity to produce 3-hydroxy-3-methylglutaryl coenzyme A (HMG-CoA) from acetoacetyl-CoA, and a DNA encoding an enzyme having the activity to produce mevalonic acid from HMG-CoA.

(5) The process according to the above item (4), wherein the DNA encoding an enzyme having the activity to produce acetoacetyl-CoA from acetyl-CoA is acetyl-CoA acetyltransferase.

(6) The process according to the above item (4), wherein the DNA encoding an enzyme having the activity to produce HMG-CoA from acetoacetyl-CoA is HMG-CoA synthase.

(7) The process according to the above item (4), wherein the DNA encoding an enzyme having the activity to produce mevalonic acid from HMG-CoA is HMG-CoA reductase.

(8) The process according to the above item (4), wherein the DNA encoding an enzyme having the activity to produce acetoacetyl-CoA from acetyl-CoA and the DNA encoding an enzyme having the activity to produce mevalonic acid from HMG-CoA are a DNA encoding acetyl-CoA acetyltransferase/HMG-CoA reductase.

(9) The process according to any one of the above items (1) to (3), wherein the DNA to be transfected is a DNA encoding acetyl-CoA acetyltransferase, a DNA encoding HMG-CoA synthase and a DNA encoding HMG-CoA reductase.

(10) The process according to any one of the above items (1) to (3), wherein the DNA to be transfected is a DNA encoding acetyl-C6A acetyltransferase/HMG-CoA reductase and HMG-CoA synthase.

(11) The process according to any one of the above items (1) to (10), wherein the DNA is derived from one or more microorganism(s) selected from the group consisting of bacteria, mycobacteria, archaebacteria and yeast.

(12) The process according to any one of the above items (1) to (10), wherein the DNA is derived from a microorganism belonging to a genus selected from the group consisting of genus Enterococcus, genus Streptococcus, genus Staphylococcus, genus Lactococcus, genus Streptomyces, genus Methanothermobacter, genus Methanococcus, genus Pyrococcus, genus Thermoplasma and genus Saccharomyces.

(13) The process according to any one of the above items (1) to (3), wherein the DNA to be transfected is one or more DNA(s) selected from the group consisting of the following (a) to (j):

(a) mvaE of Enterococcus faecalis;

(b) mvaS of Enterococcus faecalis;

(c) thiL of Lactococcus lactis;

(d) mvaA of Lactococcus lactis;

(e) hmcC of Lactococcus lactis;

(f) a DNA encoding an amino acid sequence of SEQ ID NO: 2;

(g) a DNA encoding an amino acid sequence of SEQ ID NO: 4;

(h) a DNA encoding an amino acid sequence of SEQ ID NO: 12;

(i), a DNA encoding an amino acid sequence of SEQ ID NO: 14; and

(j) a DNA encoding an amino acid sequence of SEQ ID NO: 16.

(14) The process according to the above item (3), wherein the microorganism not having a mevalonic acid pathway is a microorganism belonging to a genus selected from the group consisting of genus Escherichia, genus Serratia, genus Bacillus, genus Brevibacterium, genus Corynebacterium, genus Microbacterium, genus Pseudomonas and genus Synechocystis.

(15). The process according to the above item (14), wherein the microorganism belonging to genus Escherichia is Escherichia coli.

(16) The process according to any one of the above items (1) to (3), wherein the microorganism having the ability to biosynthesize mevalonic acid from acetyl-CoA is a microorganism obtainable by transfecting a DNA participating in biosynthesis of mevalonic acid from acetyl-CoA into a host microorganism using an expression vector.

(17) A microorganism having the ability to biosynthesize mevalonic acid from acetyl-CoA and being obtainable by transfecting a DNA participating in biosynthesis of mevalonic acid from acetyl-CoA into a host microorganism.

(18) The microorganism according to the above item (17), wherein the host microorganism is a microorganism not having the ability to biosynthesize mevalonic acid from acetyl-CoA.

(19) The microorganism according to the above item (18), wherein the microorganism not having the ability to biosynthesize mevalonic acid from acetyl-CoA is a microorganism not having a mevalonic acid pathway.

(20) The microorganism according to any one of the above items (17) to (19), wherein the DNA to be transfected is one or more DNA(S) selected from the group consisting of a DNA encoding an enzyme having the activity to produce acetoacetyl-CoA from acetyl-CoA, a DNA encoding an enzyme having the activity to produce HMG-CoA from acetoacetyl-CoA, and a DNA encoding an enzyme having the activity to produce mevalonic acid from HMG-CoA.

(21) The microorganism according to the above item (20), wherein the DNA encoding an enzyme having the activi ty to produce acetoacetyl-CoA from acetyl-CoA is acetyl-CoA acetyltransferase.

(22) The microorganism according to the above item (20), wherein the DNA encoding an enzyme.having the activity to produce HMG-CoA from acetoacetyl-CoA is HMG-CoA synthase.

(23) The microorganism according to the above item (20), wherein the DNA encoding an enzyme having the activity to produce mevalonic acid from HMG-CoA is HMG-CoA reductase.

(24) The microorganism according to the above item (20), wherein the DNA encoding an enzyme having the activity to produce acetoacetyl-CoA from acetyl-CoA and the DNA encoding an enzyme having the activity to produce mevalonic acid from HMG-CoA are a DNA encoding acetyl-CoA acetyltransferase/HMG-CoA reductase.

(25) The microorganism according to any one of the above items 17 to 19, wherein the DNA to be transfected is a DNA encoding acetyl-CoA acetyltransferase, a DNA encoding HMG-CoA .synthase and a DNA encoding HMG-CoA reductase.

(26) Themicroorganism accordingto any one of the above items. (17) to (19), wherein the DNA to be transfected is a DNA encoding acetyl-CoA acetyltransferase/HMG-CoA reductase and HMG-CoA synthase.

(27) The microorganism according to any one of the above items (17) to (26), wherein the DNA is derived from one or more microorganism(s) selected from the group consisting. of bacteria, mycobacteria, archaebacteria and yeast.

(28) The microorganism according to any one of the above items (17) to (26), wherein the DNA is derived from a microorganism belonging to a genus selected from-the group consisting of genus Enterococcus, genus Streptococcus, genus Staphylococcus, genus Lactococcus, genus Lactobacillus, genus Streptomyces, genus Methanothermiobacter, genus Methanococcus, genus Pyrococcus, genus Thermoplasma and genus Saccharomyces.

(29) The microorganism according to any one of the above items (17) to (19), wherein the DNA to be transfected is one or more DNA(S) selected from the group consisting of the following (a) to (j):

(a) mvaE of Enterococcus faecalis;

(b) mvaS of Enterococcus faecalis;

(c) thiL of Lactococcus lactis;

(d) mvaA of Lactococcus lactis;

(e) hmcc of Lactococcus lactis;

(f) a DNA encoding an amino acid sequence of SEQ ID NO: 2;

(g) a DNA encoding an amino acid sequence of SEQ ID NO: 4;

(h) a DNA encoding an amino acid sequence of SEQ ID NO: 12;

(i) a DNA encoding an amino acid sequence of SEQ ID NO: 14; and

(j) a DNA encoding an amino acid sequence of SEQ ID NO: 16.

(30) The microorganism according to the above item (19), wherein the microorganism not having a mevalonic acid pathway is a microorganism belonging to a genus selected from the group consisting of genus Escherichia, genus Serratia, genus Bacillus, genus Brevibacterium, genus Corynebacterium, genus Microbacterium, genus Pseudomonas and genus Synechocystis.

(31) The microorganism according to the above item (30), wherein the microorganism belonging to genus Escherichia is Escherichia coli.

(32) The microorganism according to any one of the above items (17) to (19), wherein the microorganism having the ability to biosynthesize mevalonic acid from-acetyl-CoA is a microorganism obtainable by transfecting a DNA participating in biosynthesis of mevalonic acid from acetyl-CoA into a host microorganism using an expression vector.

The present invention is explained below in detail.

The microorganism not having the ability to biosynthesize mevalonic acid from acetyl-CoA herein means a microorganism lacking the ability due to having a mutation on a mevalonic acid pathway or a microorganism having only a non-mevalonic acid pathway.

The mevalonic acid herein may have either one of the two types of structures, i.e., the acid form and the lactone form.

Among the microorganisms lacking the ability due to having a mutation on the mevalonic acid pathway, preferred are microorganisms having a weak activity to metabolize mevalonic acid, or not having the activity.

Examples of the microorganism having only a non-mevalonic acid pathway include bacteria belonging to genus Escherichia, genus Serratia, genus Bacillus, genus Brevibacterium, genus Corynebacterium, genus Microbacterium, genus Pseudomonas, genus Synechocystis and the like. Specifically, examples thereof include Escherichia coli, Serratia marsecense, Bacillus subtilis, Bacillus megaterium, Bacillus cereus, Bacillus amylolicuefaciens, Brevibacterium immariophilum, Brevibacterium saccharolyticum, Corynebacterium glutamicum, Corynebacterium ammoniagenes, Microbacterium ammoniaphilum, Pseudomonas putida, Pseudomonas aeruginosa, Synechocystis sp. PCC6803 and the like.

As the DNA which participates in biosynthesis of mevalonic acid from acetyl-CoA herein, any DNA may be used, so long as it encodes an enzyme that is present.in a biosynthetic pathway where mevalonic acid can be biosynthesized from acetyl-CoA. Examples of the DNA include a DNA encoding an enzyme in the reaction pathway of the mevalonic acid pathway, from acetyl-CoA until the production of mevalonic acid (hereinafter, referred to as a mevalonic acid biosynthetic enzyme), and the like.

Examples of the mevalonic acid biosynthetic enzyme include an enzyme having the activity to produce acetoacetyl-CoA from acetyl-CoA, an enzyme having the activity to produce 3-hydroxy-3-methylglutaryl coenzyme A (EMG-CoA) from acetoacetyl-CoA, and an.enzyme having the activity to produce mevalonic acid from HMG-CoA. Examples of the enzyme having the activity to produce acetoacetyl-CoA from acetyl-CoA include acetyl-CoA acetyltransferase. Examples of the enzyme having the activity to produce HMG-CoA from acetoacetyl-CoA include HMG-CoA synthase. Examples of the enzyme having the activity to produce mevalonic acid from HMG-CoA include HMG-CoA reductase. Further, also included are proteins having two or all three enzyme activities among acetyl-CoA acetyltransferase, HMG-CoA synthase and HMG-CoA reductase in a single enzyme protein, for example, acetyl-CoA acetyltransferase/HMG-CoA reductase having both enzyme activities of acetyl-CoA acetyltransferase and HMG-CoA reductase in a single enzyme.

The DNA encoding the aforementioned enzyme may be derived from any organism and may have an artificial sequence, as long as it functions as a mevalonic acid biosynthetic enzyme in the aforementioned microorganism not having the ability to biosynthesize mevalonic acid from acetyl-CoA when it is expressed in the microorganism as a host, and preferred are DNAs derived from an organism with a codon usage that is similar to the codon usage in the host microorganism, or DNAs with altered sequence such that the codon usages become similar thereto.

Hereinafter, any one of methods described in Molecular Cloning: A Laboratory Manual 3rd ed., Cold Spring Harbor Laboratory Press (2001) (hereinafter, referred to as Molecular Cloning 3rd ed.), Current Protocols in Molecular Biology, John Wiley & Sons (1987-2001) (hereinafter, referred to as Current Protocols in molecular Biology) and the like is applied as a genetic engineering procedure, unless otherwise stated.

[1] Preparation of a DNA which Participates in Biosynthesis of Mevalonic Acid from acetyl-CoA

(1) Acquisition of Nucleotide Sequence Information of a DNA Encoding a Mevalonic Acid Biosynthetic Enzyme

A DNA encoding a mevalonic acid biosynthetic enzyme can be preparedby the method mentioned in (2) below utilizing known nucleotide sequence information. Nucleotide sequence information of the DNA encoding each enzyme can be acquired by searching for the nucleotide sequence database such as GenBank.

As the DNA encoding the mevalonic acid biosynthetic enzyme which can be used for the preparation of the microorganism having the ability to biosynthesize mevalonic acid of the present invention, and the nucleotide sequence information of which is known, mentioned are genes derived from microorganisms, and the examples are acetyl-CoA acetyltransferase/HMG-CoA reductase gene mvaE and HMG-CoA synthase gene mvaS of Enterococcus faecalis, acetyl-CoA acetyltransferase/HMG-CoA reductase gene mvaE and HMG-CoA synthase gene mvaS of Enterococcus faecium, HMG-CoA reductase gene mvaA and HMG-CoA synthase gene mvaS of Streptococcus pneumoniae, HMG-CoA reductase gene. mvaA and HMG-CoA synthase gene mvaS of Streptococcus pyogenes, acetyl-CoA acetyltransferase gene thiL, HMG-CoA synthase gene hmcM and HMG-CoA reductase gene mvaA of Lactococcus lactis, acetyl-CoA acetyltransferase gene mvaC, HMG-CoA synthase gene mvaS and HMG-CoA reductase gene mvaA of Staphylococcus haemolyticus, HMG-CoA synthase gene mvaS and HMG-CoA reductase gene mvaA of Staphylococcus aureus, HMG-CoA synthase gene mvaS and HMG-CoA reductase gene mvaA of Staphylococcus epidermidis, HMG-CoA synthase gene mvaS and HMG-CoA reductase gene mvaA of Borrelia burgdorferi, HMG-CoA synthase gene hmgs and HMG-CoA reductase gene hmgr of Streptomyces griseolosporeus, HMG-CoA reductase gene MTH562 and HMG-CoA synthase gene MTH792 of Methanothermobacter thermoautotrophicum, HMG-CoA reductase gene MJ0705 and HMG-CoA synthase gene MJ1546 of Methanococcus jannaschii, HMG-CoA reductase gene PAB2106. and. HMG-CoA synthase gene PAB0906 of Pyrococcus abyssi, HMG-CoA reductase gene PH1805 and HMG-CoA synthase gene PH0677 of Pyrococcus horikoshii, HMG-CoA reductase gene Ta0406 and HMG-CoA synthase gene Tal455 of Thermoplasma acidophilum, HMG-CoA reductase gene TVN1168 and HMG-CoA synthase gene TVN0130 of Thermoplasma volcanium, and HMG-CoA synthase gene and HMG-CoA reductase gene HMG1 and HMG2 of Saccharomyces cerevisiae.

(2) Preparation of a DNA Encoding a Mevalonic Acid Biosynthetic Enzyme

A DNA encoding a mevalonic acid biosynthetic enzyme can be prepared from an organism which has a gene encoding a mevalonic acid biosynthetic enzyme and being found to have a known nucleotide sequence in (1), for example, a bacterium such as that of genus Enterococcus, genus Streptococcus, genus Staphylococcus or genus Lactococcus; a mycobacterium such as that of genus Streptomyces; an archaebacterium such as that of genus Methanothermobacter, genus Methanococcus, genus Pyrococcus or genus Thermoplasma; or a yeast such as that of genus Saccharomyces. Specifically, the enzyme can be prepared from Enterococcus faecalis, Enterococcus faecium, Streptococcus pneumoniae, Streptococcus pyogenes, Staphylococcus aureus, Staphylococcus epidermidis, Lactococcus lactis, Streptomyces griseolosporeus, Methanothermobacter thermoautotrophicum, Methanococcus jannaschii, Pyrococcus abyssi, Pyrococcus horikoshii, Thermoplasma acidophilum, Thermoplasma volcanium, Saccharomyces cerevisiae or the like.

The aforementioned microorganism is cultured in a medium which contains a carbon source, a nitrogen source, an inorganic salt and the like that can be assimilated by the microorganism, and which permits efficient proliferation of the microorganism, under the condition of the temperature and aeration suitable for growth of the microorganism. After culturing, a chromosomal DNA of the microorganism is isolated and purified according to a known method (for example, a method described in Current Protocols in Molecular Biology).

A region containing a coding region for a mevalonic acid biosynthetic enzyme is appropriately selected from the nucleotide sequence of a chromosomal DNA of the microorganism. A DNA containing at its 3′ terminal a sequence of 5′-terminal 20 to 40 bases of thenucleotide sequence of the selected region (forward primer) and a DNA containing at its 3′ terminal a complementary sequence to 3′-terminal 20 to 40 bases of the nucleotide sequence of the selected region (reverse primer) are respectively synthesized with a DNA synthesizer. A DNA encoding a mevalonic acid biosynthetic enzyme can be amplified by PCR which is carried out using these two synthetic DNAs as primers, and then can be isolated. PCR can be carried out according to a method described in Molecular Cloning 3rd ed., PCR Protocols, Academic Press (1990). Furthermore, by substituting a nucleotide such that the nucleotide sequence of the coding region for the mevalonic acid biosynthetic enzyme becomes the most suitable codon for the expression in the host microorganism, the expression level of the enzyme can be increased, and consequently, productivity of mevalonic acid can be improved. For example, when the initiation codon is not ATG but GTG, TTG or CTG, the initiation codon of the DNA encoding the mevalonic acid biosynthetic enzyme can be substituted with ATG through PCR using a forward primer in which the sequence corresponding to the initiation codon is substituted with ATG. Moreover, a codon which is rarely used in the host microorganism can be substituted with a codon which is frequently used by site-directed mutagenesis [Nucleic Acids Res., 10, 6487. (1982); Proc. Natl. Acad. Sci. USA, 79, 6409 (1982); Gene, 34; 315 (1985); Nucleic Acids Res., 13, 4431 (1985); Proc. Natl. Acad. Sci. USA, 82, 48 (1985)].

Isolated fragment is inserted into a suitable vector for E. coli as needed, and the nucleotide sequence of the fragment is determined with a DNA sequencer.

[2] Preparation of a Microorganism of the Present Invention

The microorganism having the ability to biosynthesize mevalonic acid of the present invention is obtainable as a transformant having the activity to produce mevalonic acid from acetyl-CoA by transfecting the DNA encoding the mevalonic acid biosynthetic enzyme prepared in [1] into the host microorganism described below to express the DNA therein.

When the host microorganism does not have a mevalonic acid pathway, the microorganism having the ability to biosynthesize mevalonic acid of the present invention is obtainable by transfecting three types of mevalonic acid biosynthetic. enzymes, i.e., acetyl-CoA acetyltransferase, HMG-CoA synthase and HMG-CoA reductase into the microorganism to express the DNAs therein; or by transfecting two types of mevalonic acid biosynthetic enzymes, i.e., acetyl-CoA acetyltransferase/RMG-CoA reductase and HMG-CoA synthase to express the DNAs therein.

When the host microorganism does not have the ability to biosynthesize mevalonic acid from acetyl-CoA, and endogenously has an enzyme having the activity to produce acetoacetyl-CoA from acetyl-CoA, an enzyme having the activity to produce HMG-CoA from acetoacetyl-CoA, an enzyme having the activity to produce mevalonic acid from HMG-CoA, or an enzyme having two activities among these activities in a single enzyme protein, the ability to biosynthesize mevalonic acid can be provided to the host microorganism by transfecting a DNA encoding an enzyme other than the enzyme which the host microorganism has endogenously to express the DNA therein.

When the host microorganism already has the ability to biosynthesize mevalonic acid from acetyl-CoA, the activity of the mevalonic acid biosynthetic enzyme in the host microorganism is enhanced by transfecting a DNA encoding the mevalonic acid biosynthetic enzyme prepared in [1] to express the DNA therein, and consequently, productivity of mevalonic acid can be improved.

(1) Host Microorganism

Although any microorganism can be used as the host microorganism, preferred are microorganisms having the ability to biosynthesize mevalonic acid from acetyl-CoA, microorganisms lacking the ability due to a mutation on the mevalonic acid pathway, or microorganisms having only a non-mevalonic acid pathway. Particularly preferred are microorganisms having only a non-mevalonic acid pathway, because they are believed not to have an activity to metabolize mevalonic acid.

Examples of the microorganism having only a non-mevalonic acid pathway include bacteria belonging to genus Escherichia, genus Serratia, genus Bacillus, genus Brevibacterium, genus Corynebacterium, genus Microbacterium, genus Pseudomonas and genus Synechocystis, and more specifically, include Escherichia coli (hereinafter, abbreviated as E. coli), Serratia marsecense, Bacillus subtilis, Bacillus megaterium, Bacillus cereus, Bacillus amyloliquefaciens, Brevibacterium immariophilum, Brevibacterium saccharolyticum, Corynebacterium glutamicum, Corynebacterium ammoniagenes, Microbacterium ammoniaphilum, Pseudomonas putida, Pseudomonas aeruginosa, Synechocystis sp. PCC6803 and the like.

(2) A vector Containing a DNA Encoding a Mevalonic Acid Biosynthetic Enzyme

(a) Utilization of an Expression Vector

A DNA fragment with a suitable length containing a coding region for a mevalonic acid biosynthetic enzyme is prepared from the DNA encoding a mevalonic acid biosynthetic enzyme prepared by the method mentioned in the above [1]. The DNA fragment is inserted downstream of a promoter of a suitable expression vector to construct a recombinant expression vector.

The expression vector for inserting the DNA encoding a mevalonic acid biosynthetic enzyme is preferably a recombinant DNA which can autonomously replicate in the host microorganism mentioned in (1), and which contains a promoter, a ribosome binding sequence, a restriction enzyme site for inserting the DNA encoding the mevalonic acid biosynthetic enzyme, and a transcription termination sequence. The expression vector may further contain a gene that regulates the promoter. Moreover, it is preferred that the expression vector contains a selection marker gene such as a drug resistant gene for easily selecting the transformant.

Specific examples of the expression vector include plasmids such as pKK223-3 (manufactured by Amersham Biosciences), pGEX-5X-3 (manufactured by Amersham Biosciences), pET-3a, (manufactured by Novagen), pGEMEX-1 (manufactured by Promega Corporation), pQE-30 (manufactured by QIAGEN), pKYP10 (Japanese Published Unexamined Patent Application No. 110600/1983), pKYP200 [Agric. Biol. Chem., 48, 669 (1984)), pLSA1 [Agric. Biol. Chem., 53, 277 (1989)], pGEL1 [Proc. Natl. Acad. Sci., USA, 82, 4306 (1985)], pTrS30 [prepared from E. coli JM109/pTrS30 (FERM BP-5407)], pTrS32 [prepared. from E. coli JM109/pTrS32 (FERM BP-5408)], pPA1 (Japanese Published Unexamined Patent Application No. 233798/1988), pEG400 [J. Bacteriol., 172, 2392 (1990)], pHW1520 (manufactured by MoBiTec), pRI109 (WO 00/44886) and the like.

The promoter may be any promoter as long as it can initiate the transcription of a gene positioned downstream of the promoter in the host microorganism. Examples thereof include promoters derived from E. coli, phage and the like such as trp promoter, lac promoter, P_{L} promoter, P_{R} promoter and T7 promoter, etc.; SPO1 promoter, SPO2 promoter, penp promoter and xylA promoter for expression in bacteriumof genus Bacillus; P54-6 promoter for expression in bacterium of genus Corynebacterium, and the like. In addition, artificially designed and modified promoter such. as a promoter in which two trp promoters are linked in tandem, tac promoter, lacT7 promoter or let I promoter can be also used.

It is preferred to use a vector in which the space between Shine-Dalgarno sequence, which is a ribosome binding sequence, and the initiation codon of the DNA encoding the mevalonic acid biosynthetic enzyme is adjusted to an appropriate distance (for example, 6 to 18 nucleotides). A region encoding the mevalonic acid biosynthetic enzyme and a region containing an endogenous ribosome binding sequence that exists upstream of the same in a chromosomal DNA are amplified, which may be used as a ribosome binding sequence in preparing the DNA encoding the mevalonic acid biosynthetic enzyme of [1].

Although a transcription termination sequence is not necessarily required for expression of the mevalonic acid biosynthetic enzyme, it is preferred that a transcription termination sequence is positioned immediately downstream of the DNA encoding the mevalonic acid biosynthetic enzyme (or downstream of the DNA which is positioned most downstream, when two or more DNAs encoding the mevalonic acid biosynthetic enzyme are linked downstream of one promoter).

In order to transfect two or more kinds of DNAs encoding the mevalonic acid biosynthetic enzyme into a host microorganism to express the DNAs therein, a vector which has units for the respective DNAs encoding the mevalonic acid biosynthetic enzyme is constructed; each of the units comprising a promoter, a ribosome binding sequence, a DNA encoding a mevalonic acid biosynthetic enzyme and a transcription termination sequence; or alternatively constructed is a vector,in which units for the respective DNAs are serially linked downstream of one promoter, and a transcription termination sequence is present downstream of the last unit, each of the units comprising a ribosome binding sequence and a DNA encoding a mevalonic acid biosynthetic enzyme.

Examples of the expression vector containing a DNA encoding a mevalonic acid biosynthetic enzyme constructed as described above include pMV1 containing an acetyl-CoA acetyltransferase/HMG-CoA reductase gene mvaE of Enterococcus faecalis, pMV2.containing mvaE and an HMG-CoA synthase gene mvaS of Enterococcus faecalis, pMV4 containing an acetyl-CoA acetyltransferase gene thiL and an HMG-CoA reductase gene mvaA of Lactococcus lactis, and pMV5 containing thiL, mvaA and an HMG-CoA synthase gene hmcM of Lactococcus lactis.

(b) Expression by Endogenous Promoter

When a region encoding the mevalonic acid biosynthetic enzyme in the chromosomal DNA together with a region containing an endogenous ribosome binding sequence and a promoter that are present upstream thereof are amplified in preparing the DNA encoding the mevalonic acid biosynthetic enzyme of [1], the mevalonic acid biosynthetic enzyme can be expressed without using the abovementioned expression vector by means of the endogenous promoter by transfecting the DNA into a host microorganism. For the transfection, a recombinant vector is constructed by inserting the DNA into a vector which can autonomously replicate in a host microorganism and contains a restriction enzyme site for inserting the DNA and a selection marker gene such as a drug resistant gene for easily selecting the transformant. Examples of the vector which may be used for inserting the DNA include pBR322 [Gene, 2, 95 (1977)], pBluescript II KS (+) (manufactured by Stratagene), pUC18 [Gene, 33, 103 (1985)], pHY300PLK (manufactured by Takara Shuzo Co., Ltd.), and the like.

(3) Transfection of a Vector Containing a DNA Encoding Mevalonic Acid Biosynthetic Enzyme into a Host Microorganism and Preparation of a Transformant

As a method for transfecting the vector containing the DNA encoding a mevalonic acid biosynthetic enzyme constructed in (2) into a host microorganism, any method for transfecting a DNA into a host microorganism mentioned in (1) can be used, and examples thereof include a method in which a calcium ion is used [Proc. Natl. Acad. Sci., USA, 69, 2110 (1972)], a protoplast method (Japanese Published Unexamined Patent Application No. 248394/1988), an electroporation method [Nucleic Acids Research, 16, 6127 (1988)], and the like. The host microorganism transfected with the vector is cultured, and the transformant is obtainable through selecting on the basis of the change of the phenotype (resistance against a drug, or the like) according to the selection marker in the expression vector.

Examples of the recombinant microorganism having the ability to biosynthesize mevalonic acid prepared as described hereinabove include E. coli strain XL1-Blue/pMV2 which is prepared by transfecting pMV2, an expression vector of acetyl-CoA acetyltransferase/HMG-CoA reductase and HMG-CoA synthase, into E. coli XL1-Blue, and E. coli strain NM522/pMV5 which is prepared by transfecting pMV5, an expression vector of acetyl-CoA acetyltransferase, HMG-CoA reductase and HMG-CoA synthase, into E. coli NM552.

[3] Production of Mevalonic Acid

(1) Culture of a Recombinant Microorganism

Medium for culturing the recombinant microorganism obtainable by the method described in [2] above may be either a natural medium or a synthetic medium, as long as it is a medium which contains a carbon source, a nitrogen source, an inorganic salt and the like that can be assimilated by the microorganism, and which permits efficient culture of the microorganism.

The carbon source may be any carbon source which can be assimilated by the microorganism, and examples thereof include carbohydrates such as glucose, fructose, sucrose, molasses containing the same and starch or starch hydrolysate, organic acids such as acetic acid and propionic acid, alcohols such as ethanol and propanol, and the like.

Examples of the nitrogen source include ammonium salts of an inorganic acid or organic acid such as ammonia, ammonium chloride, ammonium sulfate, ammonium acetate and ammonium phosphate, other nitrogenous compounds, peptone, meat extract, yeast extract, corn steep liquor, casein hydrolysate, soy bean cake and soy bean cake hydrolysate, microbial body used for the fermentation and the digestion product of the same, and the like.

Examples of the inorganic salt include potassium dihydrogenphosphate, dipotassium hydrogenphosphate, magnesium phosphate, magnesium sulfate, sodium chloride, ferrous sulfate, manganese sulfate, copper sulfate, calcium carbonate and the like.

An antibiotic such as ampicillin or tetracycline may be added to the medium, if necessary. Further, when a microorganism transformed with an expression vector containing an inducible promoter is cultured, the inducer for the promoter may be added to the medium during the cultivations, if necessary. Examples of the inducer include isopropyl-β-D-thiogalactoside for lac promoter, indoleacrylic acid for trp promoter, and the like.

Cultivation is usually conducted under an aerobic condition, by shaking culture or aerating with stirring culture. The cultivation temperature is preferable 15 to 40° C., and the cultivation time is usually 5 hours to 7 days. During the cultivation, pH is kept at 3.0 to 9.0. The pH is adjusted using an inorganic acid or an organic acid, an alkaline solution, urea, calcium carbonate, aqueous ammonia or the like.

(2) Recovery of Mevalonic Acid from Culture

Mevalonic acid can be recovered from the culture medium using an isolation and purification method which is generally employed in organic chemistry such as extraction with an organic solvent, crystallization, thin layer chromatography and high performance liquid chromatography.

The recovered mevalonic acid can be subjected to structural verification or quantitative determination by ¹³C-NMR spectrum, ¹H-NMR spectrum, mass spectrum, gas chromatography, high performance liquid chromatography or the like.

Examples of the present invention are shown below, however the present invention is not limited to these Examples.

### BRIEF DESCRIPTION OF THE DRAWINGS

FIG. 1 shows a construction step of pMV1.

FIG. 2 shows a construction step of pMV2.

FIG. 3 shows a construction step of pMV4.

FIG. 4 shows a construction step of pMV5.

[Description of Abbreviation┘
Ampr: ampicillin resistant gene
Ptrp: trp promoter
mvaE: acetyl-CoA acetyltransferase/HMG-CoA reductase gene
mvaS :HMG-CoA synthase gene
thiL: acetyl-CoA acetyltransferase gene
mvaA: HMG-CoA reductase gene
hmcM: HMG-CoA synthase gene

### BEST MODE FOR CARRYING OUT THE INVENTION

### EXAMPLE 1

### Search for Genes on Database

Using acetyl-coenzyme A acetyltransferase, 3-hydroxy-3-methylglutaryl-coenzyme A synthase and 3-hydroxy-3-methylglutaryl coenzyme A reductase as a query, key word search was carried out on the nucleotide sequence database GenBank.

As a consequence, sequence information was obtained as to the following enzyme genes derived from the bacteria, i.e., acetyl-CoA acetyltransferase/HMG-CoA reductase gene. mvaE (GenBank Accession No.: AF290092) and HMG-CoA-synthase gene mvaS (GenBank Accession No.: AF290092) of Enterococcus faecalis, acetyl-CoA acetyltransferase/HMG-CoA reductase gene mvaE (GenBank. Accession No.: AF290094) and HMG-CoA synthase gene mvaS (GenBank -Accession. No.: AF290094) of Enterococcus faecium, HMG-CoA reductase gene mvaA (GenBank Accession No.: AF290098) and HMG-CoA synthase gene mvaS (GenBank Accession No.: AF290098) of Streptococcus pneumoniae, HMG-CoA reductase gene mvaA (GenBank Accession No.: AF290096) and HMG-CoA synthase gene mvaS (GenBank Accession No.: AF290096) of Streptococcus pyogenes, acetyl-CoA acetyltransferase gene thiL (GenBank Accession No.: AE006387), HMG-CoA synthase gene hmcM (GenBank Accession No.: AE006388) and HMG-CoA reductase gene mvaA (GenBank Accession No.: AE006387) of Lactococcus lactis, acetyl-CoA acetyltransferase gene mvaC (GenBank Accession No.: AF290088), HMG-CoA synthase gene mvaS (GenBank Accession No.: AF290088), and HMG-CoA reductase gene mvaA (GenBank Accession No.: AF290088) of Staphylococcus haemolyticus, HMG-CoA synthase gene mvaS (GenBank Accession No.: AF290086) and HMG-CoA reductase gene mvaA (GenBank Accession No.: AF290086) of Staphylococcus aureus, HMG-CoA synthase gene mvaS (GenBank Accession No.: AF290090) and HMG-CoA reductase gene mvaA (GenBank Accession No.: AF290090) of Staphylococcus epidermidis, HMG-CoA synthase gene BB0683 (GenBank Accession No.: AE001169) and HMG-CoA reductase gene BB0685 (GenBank Accession No.: AE001169) of Borrelia burgdorferi and the like; as to the following enzyme genes derived from the mycqbacteria, i.e., HMG-CoA synthase gene hmgs (GenBank Accession No.: AB037907) and HMG-CoA reductase gene-hmqr (GenBank Accession No.: AB037907) of Streptomyces griseolosporeus and the like; as to the following enzyme genes derived from the archaebacteria, i.e., HMG-CoA reductase gene MTH562 (GenBank Accession No.: NC_{—}000916) and HMG-CoA synthase gene MTH792 (GenBank Accession No.: NC_{—}000916) of Methanothermobacter thermoautotrophicum, HMG-CoA reductase gene MJ0705 (GenBank Accession No.: NC_{—}000909) and HMG-CoA synthase gene MJ1546 (GenBank Accession No.: NC_{—}000909) of Methanococcus jannaschii, HMG-CoA reductase gene PAB2106 (GenBank Accession No.: NC_{—}000868) and HMG-CoA synthase gene PAB0906 (GenBank Accession No.: NC_{—}000868) of Pyrococcus abyssi, HMG-CoA reductase gene PH1805 (GenBank Accession No.: NC_{—}000961) and HMG-CoA synthase gene PH0677 (GenBank Accession No.: NC_{—}000961) of Pyrococcus horikoshii, HMG-CoA reductase gene Ta0406 (GenBank Accession No.: AL445064) and HMG-CoA synthase gene Ta1455 (GenBank Accession No.: NC 002578) of Thermoplasma acidophilum, HMG-CoA reductase gene TVN1168 (GenBank Accession No.: NC_{—}002689) and HMG-CoA synthase gene TVN0130 (GenBank Accession No.: NC_{—}002689) of Thermoplasma volcanium and the like; as to the following enzyme genes derived from the yeast, i.e., HMG-CoA synthase gene (GenBank Accession No.: X96617) and HMG-CoA reductase gene HMG1 (GenBank Accession No.: NC_{—}001145) and HMG2. (GenBank Accession No.:. NC_{—}001144) of Saccharomyces cerevisiae and the like; and as to the enzyme genes derived from higher eukaryote such as mouse, human or Arabidopsis thaliana.

### EXAMPLE 2

### Preparation of E. coli Strain that Expresses Mevalonic Acid Biosynthetic Enzyme Genes Derived from Enterococcus faecalis

(1) Isolation of mvaE and mvas Derived from Enterococcus faecalis

Among the sequence information obtained in Example 1, a sequence (GenBank Accession No.: AF290092) containing the gene mvaE encoding acetyl-CoA acetyltransferase/HMG-CoA reductase and the gene mvaS encoding HMG-CoA synthase of Enterococcus faecalis (hereinafter, abbreviated as E. faecalis) was utilized to obtain the gene fragments as described below. A nucleotide sequence of mvaE of E. faecalis (hereinafter, merely described as mvaE) is shown in SEQ ID NO: 1; an amino acid sequence of acetyl-CoA acetyltransferase/HMG-CoA reductase encoded by the gene is shown in SEQ ID NO: 2; a nucleotide sequence of mvas of E. faecalis (hereinafter, merely described as mvaS) is shown in SEQ ID NO: 3; and an amino acid sequence of HMG-CoA synthase encoded by the gene is shown in SEQ ID NO: 4.

First, E. faecalis (ATCC 14508) was inoculated into a brain heart infusion broth (manufactured by Difco), and subjected to static culture overnight at 30° C. After the cultivation, a chromosomal DNA of the microorganism was isolated and purified according to a method in which saturated phenol is used, as described in Current Protocols in Molecular Biology.

Using 8905 DNA synthesizer manufactured by Perseptive Biosystems, Inc., DNAs having the sequences shown in SEQ ID NO: 5 to 8, respectively, (hereinafter, referred to as primer A, primer B, primer C, primer D, respectively) were synthesized. The sequence of SEQ ID NO: 5 corresponds to a sequence of positions 1 to 24 of the sequence of SEQ ID NO: 9 (partial sequence of the chromosomal DNA of E. faecalis containing mvaE), in which the initiation codon (ttg) of mvaE is substituted with atg, and a sequence containing a ClaI recognition sequence is added at its 5′ terminal. The sequence of SEQ ID NO: 6 corresponds to a sequence complementary to the sequence of positions 2395 to 2418 of the sequence of SEQ ID NO: 9, in which a sequence containing a BamHI recognition sequence is added at its 5′ terminal. The sequence of SEQ ID NO: 7 corresponds to a sequence of positions 1 to 20 of the sequence of SEQ ID NO: 10 (partial sequence of the chromosomal DNA of E. faecalis containing MvaS), in which a sequence containing a BglII recognition sequence is added at its 5′ terminal. The sequence of SEQ ID NO: 8 corresponds to a sequence complementary to the sequence of positions 1144 to 1166 of the sequence of SEQ ID NO: 10, in which a sequence containing a BamHI recognition sequence is added at its 5′ terminal.

Using the chromosomal DNA of E. faecalis as a template, PCR was conducted using the aforementioned primer A and primer B for amplification of the mvaE fragment, or the primer C and primer D for amplification of the mvaS fragment, respectively. PCR was conducted by subjecting 40 μL of a reaction solution containing 0.1 μg of the chromosomal DNA, 0.5 μmol/L of each primer, 2.5 units of Pfu DNA polymerase (manufactured by Stratagene), 4 μL of ×10 buffer for Pfu DNA polymerase (manufactured by Stratagene) and 200 μmol/L of each dNTP (dATP, dGTP, dCTP and TTP) to the cycles of at 94° C. for 1 min, at 55° C. for 2 min and at 72° C. for 3 min repeated 30 times.

Aliquot of each reaction solution in an amount of 1/10 was subjected to an agarose gel electrophoresis, and after the confirmation that a fragment of about 2.4 kb corresponding to the mvaE fragment was amplified by PCR using primer A and primer B and that a fragment of about 1.1 kb corresponding to the mvaS fragment was amplified by PCR using primer C and primer D, each residual reaction solution was mixed with an equal volume of TE [10 mmol/L Tris-HCl (pH 8.0), 1 mmol/L EDTA] saturated phenol/chloroform (1 vol/1 vol). Eachmixturewas centrifuged, and the resulting upper layer was mixed with two fold volume of cold ethanol. Each mixture was left to stand at −80° C. for 30 min, and centrifuged to give a precipitate of the DNA. The precipitate of each DNA was dissolved in 20 μL of TE.

Using each 5 μL of the solution, the DNA amplified with primer A and primer B was cleaved with restriction enzymes ClaI and BamHI, while the DNA amplified with primer C and primer D was cleaved with restriction enzymes BglII and BamHI. After separating the DNA fragments by agarose gel electrophoresis, DNA fragments of 2.4 kb containing mvaE and of 1.1 kb containing MvaS were recovered, respectively, with GENECLEAN II kit, (manufactured by BIO 101, Inc.).

(2) Construction of an Expression Vector pMV1 Containing mvaE

After 0.2 μg of an expression vector pTrs30 [which can be prepared from E. coli JM109/pTrS30 (FERM BP-5407)] containing a trp promoter was cleaved with restriction enzymes claI and BamHI, the DNA fragments were separated by agarose gel electrophoresis, and a DNA fragment of 4.6 kb was similarly recovered.

Using the fragment of 2.4 kb containing mvaE prepared in (1) and the fragment of 4.6 kb, and a ligation kit (manufactured by Takara Shuzo Co., Ltd.), a ligation reaction was conducted at 16° C. for 16 hrs.

E. coli strain XL1-Blue (manufactured by Stratagene) was transformed with the ligation reaction solution according to the method in which a calcium ion is used [Proc. Natl. Acad. Sci., USA, 69, 2110 (1972)]. The transformant was inoculated on an LB agar medium (10 g/L triptone, 5 g/L yeast extract, 10 g/L sodium chloride, 15 g/L agar,. pH 7.0) containing 50 μg/mL ampicillin, and cultured at 30° C. overnight.

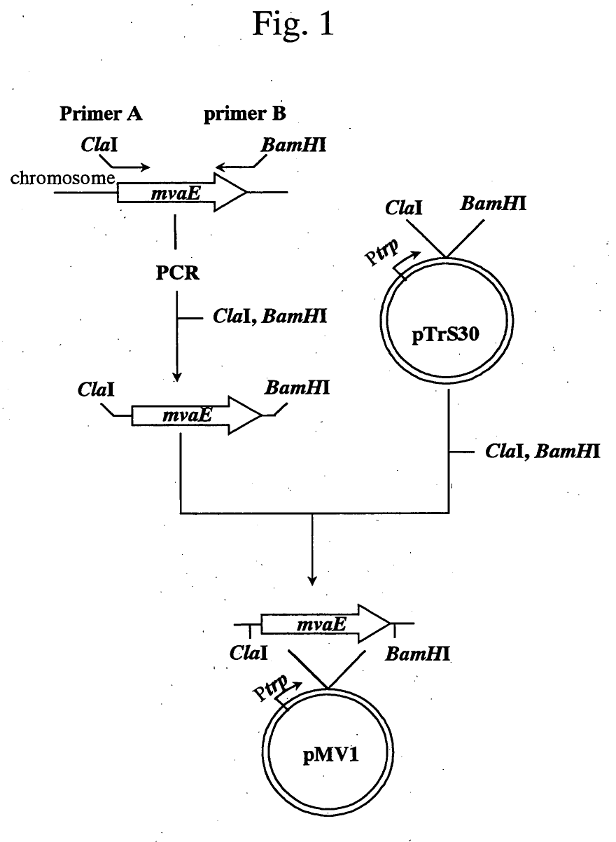

Plasmid was extracted from the colony of the grown transformant according to a known method to obtain pMV1 that is an expression vector of acetyl-CoA acetyltransferase/HMG-CoA reductase containing mvaE. Structure of the vector was determined by restriction enzyme digestion. The construction step as described hereinabove is illustrated in FIG. 1.

(3) Construction of an Expression Vector pMV2 Containing each Gene Fragment of mvaE and MvaS

After cleaving pMV1 with a restriction enzyme BamHI, the DNA fragments were separated by agarose gel electrophoresis, and a DNA fragment of 7 kb was recovered with GENECLEAN II kit. Dephosphorylation of ends of the DNA fragment was carried out with a blunt DNA kit (manufactured by Takara Shuzo Co., Ltd.).

Using the fragment of 7 kb and the fragment of 1.1 kb containing MvaS, and the ligation kit, a ligation reaction was conducted at 16° C. for 16 hrs.

E. coli strain XL1-Blue was transformed with the ligation reaction solution according to the aforementioned method. The transformant was inoculated on an LB agar medium containing 50 μg/mL ampicillin, and cultured at 30° C. overnight.

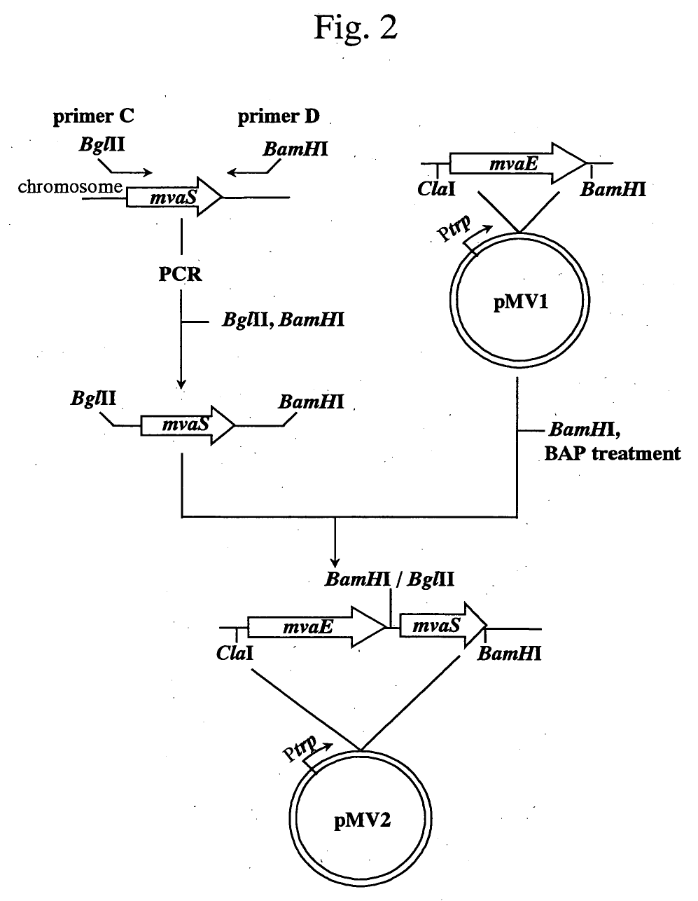

Plasmid was extracted from the colony of the grown transformant according to the aforementioned method, and the structure of the plasmid was determined by restriction enzyme digestion. As a result, it was confirmed that pMV2, an expression vector having a structure with mvaE and MvaS inserted in a forward direction of their transcription from the trp promoter, was obtained. The construction step as described hereinabove is illustrated in FIG. 2. pMV2 is an expression vector of acetyl-CoA acetyltransferase/HMG-CoA reductase and HMG-CoA synthase. The transformant containing pMV2 is designated as E. coli strain XL1-Blue/pMV2.

### EXAMPLE 3

### Production of Mevalonic Acid by E. coli Strain XL1-Blue/pMV2

(1) Production of Mevalonic Acid by Culturing in a Test Tube

The E. coli strain XL1-Blue/pMV2 obtained in Example 2 was inoculated into 8 mL of an LB medium (10 g/L triptone, 5 g/L yeast extract, 10 g/L sodium chloride, pH 7.0) containing 50 μg/mL ampicillin in a large test tube, and cultured at 28° C. for 17 hrs. The culture medium was inoculated into 8 mL of an M9X medium [16 g/L dipotassium hydrogenphosphate, 14 g/L potassium dihydrogenphosphate, 5 g/L ammonium sulfate, 1 g/L citric acid (anhydride), 5 g/L peptone (manufactured by Kyokuto Pharmaceutical Industrial Co., Ltd.), 20 g/L glucose, 0.8 mg/L vitamin B₁, 25 mg/L magnesium sulfate heptahydrate, 50 mg/L ferrous sulfate heptahydrate, pH 7.2, adjusted with 10 mol/L sodium hydroxide: Glucose, vitamin B₁, magnesium sulfate heptahydrate and ferrous sulfate heptahydrate were separately added after autoclaving] containing 100 μg/mL ampicillin in a large test tube at 1%, and cultured at 30° C. for 48 hrs. The culture medium was centrifuged to prepare the culture supernatant. As a control, a transformant E. coli strain XL1-Blue/pTrS30 which is a transformant containing a vector pTrS30 was similarly cultured, and the culture supernatant was prepared.

The culture product in each culture supernatant was analyzed with gas chromatography according to the method described in Agr. Biol. Chem. 37, 1013 (1973). It was confirmed that 3.9 g/L mevalonic acid was produced and accumulated in the culture medium of the E. coli strain XL1-Blue/pMV2. On the other hand, production of mevalonic acid was not found in the E. coli strain XL1-Blue/pTrS30 containing the vector pTrS30 alone.

(2) Production of Mevalonic Acid using a 2 L Jar Fermenter

The E. coli strain XL1-Blue/pMV2 obtained in Example 2 was inoculated into 50 mL of an LB medium containing. 100 μg/mL ampicillin in an Erlenmeyer flask, and cultured at 28° C. for 17 hrs.

The culture medium was inoculated into 950 mL of an M9XA medium [7 g/L potassium dihydrogenphosphate, 5 g/L ammonium chloride, 1 g/L citric acid (anhydride), 1 g/L peptone (manufactured by Kyokuto Pharmaceutical Industrial Co., Ltd.), 10 mg/L manganese sulfate heptahydrate, 20 g/L glucose, 10 mg/L vitamin B₁, 2 g/L magnesium sulfate heptahydrate, 0.2 g/L ferrous-sulfate heptahydrate, pH 7.2, adjusted with 10 mol/L sodium hydroxide: Glucose, vitamin B₁, magnesium sulfate heptahydrate and ferrous sulfate heptahydrate were separately added after autoclaving] in a 2 L jar fermenter at 1%. During the cultivation, pH of the medium was kept at pH 7.0 with 18% aqueous ammonia. Culturing was conducted by stirring at 900 rpm and aeration with compressed air at 1.0 vvm which was sterilized via a filter for sterilization. Culturing was conducted at a temperature of 30° C. After depletion of the initially added sugar (after 24 hours), a sterilized 60% glucose solution was supplied at a rate of 10 to 13 mL per hour.

The culture product in the culture supernatant was analyzed with gas chromatography, and it was confirmed that 50.2 g/L mevalonic acid was produced and accumulated in the culture medium after 50 hours of cultivation.

### EXAMPLE 4

### Preparation of E. coli Strain that Expresses Mevalonic Acid Biosynthetic Enzymes Derived from Lactococcus lactis

(1) Isolation of DNA Fragments Containing of thiL Gene, mvaA Gene and hmcM Gene Respectively Derived from Lactococcus lactis

Among the sequence information obtained in Example 1, the sequence (GenBank Accession No.: AE006387) containing the acetyl-CoA acetyltransferase gene thiL and HMG-CoA reductase gene mvaA of Lactococcus lactis (hereinafter, abbreviated as L. lactis), and the sequence (GenBank Accession No.: AE006388) containing the HMG-CoA synthase gene hmcM of L. lactis were utilized to prepare these gene fragments as follows.

A nucleotide sequence of thiL of L. lactis (hereinafter, merely described as thiL) is shown in SEQ ID NO: 11; an amino acid sequence of acetyl-CoA acetyltransferase encoded by the gene is shown in SEQ ID NO: 12; a nucleotide sequence of mvaA of L. lactis (hereinafter, merely described as mvaA) is shown in SEQ ID NO: 13; an amino acid sequence of HMG-CoA reductase encoded by the gene is shown in SEQ ID NO: 14; hmcM of L. lactis (hereinafter, merely described as hmcM) is shown in SEQ ID NO: 15; and an amino acid sequence of HMG-CoA synthase encoded by the gene is shown in SEQ ID NO: 16.

L. lactis (ATCC No. 13675) was inoculated into a brain heart infusion broth (manufactured by Difco), and subjected to static culture overnight at 30° C. After the cultivation, a chromosomal DNA of the microorganism was isolated and purified according to a method in which saturated phenol is used, as described in Current Protocols in Molecular Biology.

Using 8905 DNA synthesizer manufactured by Perseptive Biosystems, Inc., DNAs having the sequences of SEQ ID NO: 17 to 20, respectively, (hereinafter, referred to as primer E, primer F, primer G, primer H, respectively) were synthesized.

The sequence of SEQ ID NO: 17 corresponds to a sequence of positions 1 to 21 of the sequence of SEQ ID NO: 21 (partial sequence of the chromosomal DNA of L lactis containing thiL and mvaA), in which the initiation codon (gtg) of thiL is substituted with atg and a sequence containing a ClaI recognition sequence is added at its 5′ terminal. The sequence of SEQ ID NO: 18 corresponds to a sequence complementary to the sequence of positions 2444 to 2464 of the sequence of SEQ ID NO: 21, in which a sequence containing a BamHI recognition sequence is added at its 5′ terminal. The sequence of SEQ ID NO: 19 corresponds to a sequence of positions 1 to 17 of the sequence of SEQ ID NO: 22 (partial sequence of the chromosomal DNA of L lactis containing hmcM), in which a sequence containing a BglII recognition sequence is added at its 5′ terminal. The sequence of SEQ ID NO: 20 corresponds to a sequence complementary to the sequence of positions 1159 to 1179 of the sequence of SEQ ID NO: 22, in which a sequence containing a BamHI recognition sequence is added at its 5′ terminal.

Using the chromosomal DNA of L. lactis as a template, PCR was conducted using the aforementioned primer E and primer F for amplification of thiL and mvaA, and using primer G and primer H for amplification of hmcM, respectively. PCR was conducted by subjecting 40 μL of a reaction solution containing 0.1 μg of the chromosomal DNA, 0.5 μmol/L of each primer, 2.5 units of Pfu DNA polymerase (manufactured by Stratagene), 4 μL of ×10 buffer for Pfu DNA polymerase (manufactured by Stratagene) and 200 μmol/L of each dNTP to the cycles of at 94° C. for 1 min, at 55° C. for 2 min and at 72° C. for 3 min repeated 30 times.

Aliquot of each reaction solution in an amount of 1/10 was subjected to on an agarose gel electrophoresis, and after the confirmation that a fragment of about 2.5 kb containing thiL and mvaA was amplified by PCR using primer E and primer F and that a fragment of about 1.2 kb containing hmcM was amplified by PCR using primer G and primer H, each residual reaction mixture was mixed with an equal volume of TE saturated phenol/chloroform. Each mixture was centrifuged, and the resulting upper layer was mixed with two-fold volume of cold ethanol. Each mixture was left to stand at −80° C. for 30 min, and centrifuged to give a precipitate of the DNA. The precipitate of each DNA was dissolved in 20 μL of TE.

Using 5 μL of the solution, the DNA amplified with primer E and primer F was cleaved with restriction enzymes ClaI and BamHI, while the DNA amplified with primer G and primer H was cleaved with restriction enzymes BglII and BamHI. After separating the DNA fragments by agarose gel electrophoresis, DNA fragments of 2.5 kb containing thiL and mvaA and of 1.2 kb containing hmcM were recovered, respectively, with GENECLEAN II kit.

(2) Construction of an Expression Vector pMV4 Containing thiL and mvaA

Using the fragment of 2.5 kb containing thiL and mvaA prepared in (1) and the ClaI-BamHI fragment (4.6 kb) of pTrS30 prepared in Example 2(2), and a ligation kit (manufactured by Takara Shuzo Co., Ltd.), a ligation reaction was conducted at 16° C. for 16 hrs.

E. coli strain NM522 [ATCC No. 47000, J. M. Biol.; 166, 1 (1983)] was transformed with the ligation reaction solution according to the method described above. The transformant was inoculated on an LB. agar medium containing 50 μg/mL ampicillin, and cultured at 30° C. overnight.

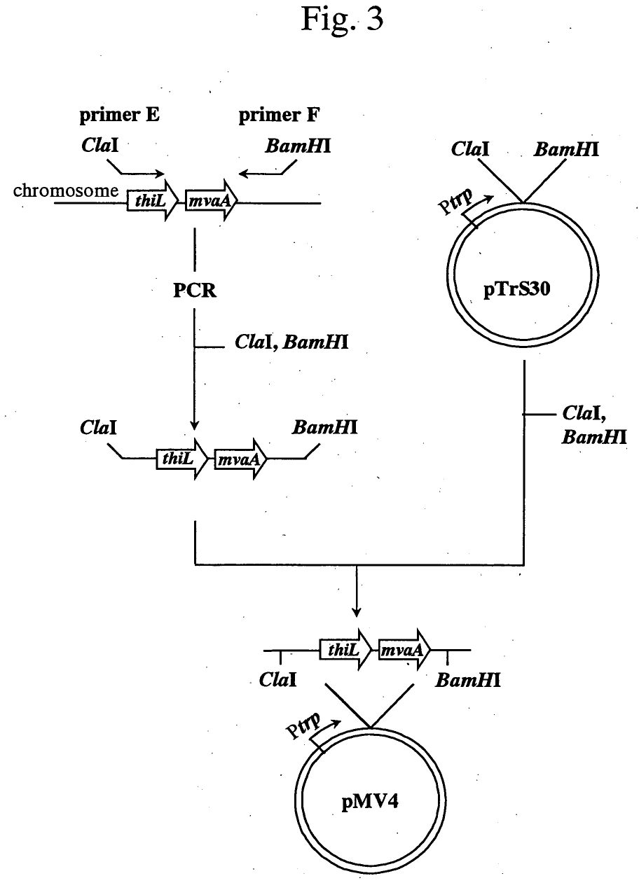

Plasmid was extracted from the colony of the grown transformant according to the method described above to obtain pMV4 that is an expression vector containing thiL and mvaA. pKV4 is an expression vector of acetyl-CoA acetyltransferase and HMG-CoA reductase. Structure of the vector was determined by restriction enzyme digestion. The construction step as described hereinabove is illustrated in FIG. 3.

(3) Construction of an Expression Vector pMV5 Containing thiL, mvaA and hmcM

Ater cleaving pMV4 with a restriction enzyme BamHI, the DNA fragments were separated by agarose gel electrophoresis, and a DNA fragment of 7 kb was recovered with GENECLEAN II kit. Dephosphorylation of ends of the fragment was carried out with ablunt DNA kit (manufactured by Takara Shuzo Co., Ltd.). Using the fragment of 7 kb and the fragment of 1.2 kb containing hmcM prepared in (1), and the ligation kit, a ligation reaction was conducted at 16° C. for 16 hrs.

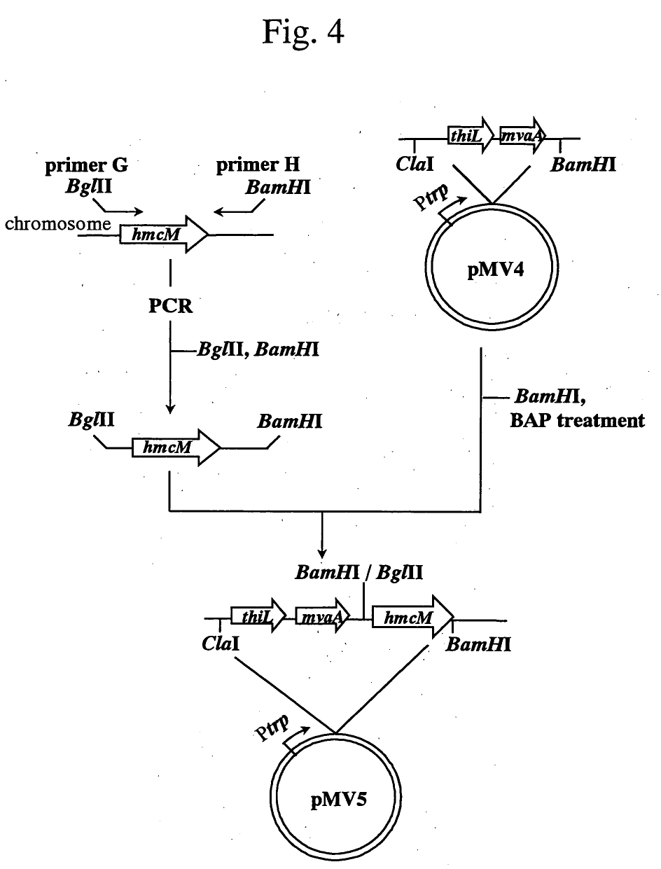

E. coli strain NM522 was transformed with the ligation reaction solution according to the aforementioned method. The transformant was inoculated on an LB agar medium containing 50 μg/mL ampicillin, and cultured at 30° C. overnight. Plasmid was extracted from the colony of the grown transformant according to the aforementioned known method, and the structure of the plasmid was determined by restriction enzyme digestion. As a result, it was confirmed that pMV5, an expression vector having a structure with thiL, mvaA and hmcC inserted in a forward direction of their transcription from the trp promoter, was obtained. The construction step as described hereinabove is illustrated in FIG. 4. pMV5 is an expression vector of acetyl-CoA acetyltransferase, HMG-CoA reductase and HMG-CoA synthase. The transformant containing pMV5 is designated as E. coli strain NM522/pMV5.

### EXAMPLE 5

### Production of Mevalonic Acid by E. coli Strain NM522/pMV5

(1) Production of Mevalonic Acid by Culturing in a Test Tube

The E. coli strain NM522/pMV5 obtained in Example 4 was inoculated into 8 mL of an LB medium containing 50 μg/mL ampicillin in a large test tube, and cultured at 28° C. for 17 hrs. The culture medium was inoculated into 8 mL of an M9X medium containing 100 μg/mL ampicillin in a large test tube at 1%, and cultured at 30° C. for 48 hrs. The culture medium was centrifuged to prepare the culture supernatant. As a control, a transformant E. coli NM522/pTrS30 strain containing a vector pTrS30 was similarly cultured to prepare the culture supernatant.

The culture product in each culture supernatant was analyzed with gas chromatography according to the method described above. It was confirmed that 1.0 g/L mevalonic acid was produced and accumulated in the culture fluid. On the other hand, production of mevalonic acid was not found in the E. coli strain NM522/pTrS30 containing the vector pTrS30 alone.

(2) Production of Mevalonic Acid using a 2 L Jar Fermenter

The E. coli strain NM522/pMV5 obtained in Example 4 was inoculated into 50 mL of an LB medium containing 100 μg/mL ampicillin in an Erlenmeyer flask, and cultured at 28° C. for 17 hrs.

The culture fluid was inoculated into 950 mL of an M9XA medium in a 2 L jar fermenter at 1%. During the cultivation, pH of the medium was kept at pH 7.0 with 18% aqueous ammonia. Culturing was conducted by stirring at 900 rpm and aeration with compressed air at 1.0 vvm which was sterilized via a filter for sterilization. Culturing was conducted at a temperature of 30° C. After depletion of the initially added sugar (after 24 hours), a sterilized 60% glucose solution was supplied at a rate of 10 to 13 mL per hour.

The culture product in the culture supernatant was analyzed with gas chromatography, and it was confirmed that 15.3 g/L mevalonic acid was produced and accumulated in the culture medium after 50 hours of cultivation.

### INDUSTRIAL APPLICABILITY

According to the present invention, mevalonic acid can be efficiently produced from an inexpensive raw material.

[Sequence Listing Free Text]

SEQ ID NO: 1—inventor: Tabata, Kazuhiko; Hashimoto, Shinichi
SEQ ID NO: 5—primer A
SEQ ID NO: 6—primer B
SEQ ID NO: 7—primer C
SEQ ID NO: 8—primer D
SEQ ID NO: 17—primer E
SEQ ID NO: 18—primer F
SEQ ID NO: 19—primer G
SEQ ID NO: 20—primer H

1. A process for producing mevalonic acid, which comprises:
culturing a microorganism having the ability to biosynthesize mevalonic acid from acetyl coenzyme A (acetyl-CoA) in a medium, the microorganism being obtainable by transfecting a DNA participating in biosynthesis of mevalonic acid from acetyl-CoA into a host microorganism;
allowing production and accumulation of mevalonic acid in the culture; and
recovering mevalonic acid from the culture.

2. The process according to claim 1, wherein the host microorganism is a microorganism not having the ability to biosynthesize mevalonic acid from acetyl-CoA.

3. The process according to claim 2, wherein the microorganism not having the ability to biosynthesize mevalonic acid from acetyl-CoA is a microorganism not having a mevalonic acid pathway.

4. The process according to any one of claims 1 to 3, wherein the DNA to be transfected is one or more DNA(s) selected from the group consisting of a DNA encoding an enzyme having the activity to produce acetoacetyl-CoA from acetyl-CoA, a DNA encoding an enzyme having the activity to produce 3-hydroxy-3-methylglutaryl coenzyme A (HMG-CoA) from acetoacetyl-CoA, and a DNA encoding an enzyme having the activity to produce mevalonic acid from HMG-CoA.

5. The process according to claim 4, wherein the DNA encoding an enzyme having the activity to produce acetoacetyl-CoA from acetyl-CoA is a DNA encoding acetyl-CoA acetyltransferase.

6. The process according to claim 4, wherein the DNA encoding an enzyme having the activity to produce HMG-CoA from acetoacetyl-CoA is a DNA encoding HMG-CoA synthase.

7. The process according to claim 4, wherein the DNA encoding an enzyme having the activity to produce mevalonic acid from HMG-CoA is a DNA encoding HMG-CoA reductase.

8. The process according to claim 4, wherein the DNA encoding an enzyme having the activity to produce acetoacetyl-CoA from acetyl-CoA and the DNA encoding an enzyme having the activity to produce mevalonic acid from HMG-CoA are a DNA encoding acetyl-CoA acetyltransferase/HMG-CoA reductase.

9. The process according to any one of claims 1 to 3, wherein the DNA to be transfected is a DNA encoding acetyl-CoA acetyltransferase, a DNA encoding HMG-CoA synthase and a DNA encoding HMG-CoA reductase.

10. The process according to any one of claims 1 to 3, wherein the DNA to be transfected is a DNA encoding acetyl-CoA acetyltransferase/HMG-CoA reductase and a DNA encoding HMG-CoA synthase.

11. The process according to claim 8, wherein the DNA is derived from one or more microorganism(s) selected from the group consisting of bacteria, mycobacteria, archaebacteria and yeast.

12. The process according to claim 8, wherein the DNA is derived from a microorganism belonging to a genus selected from the group consisting of genus Enterococcus, genus Streptococcus, genus Staphylococcus, genus Lactococcus, genus Streptomyces, genus Methanothermobacter, genus Methanococcus, genus Pyrococcus, genus Thermoplasma and genus Saccharomyces.

13. The process according to any one of claims 1 to 3, wherein the DNA to be transfected is one or more DNA(s) selected from the group consisting of the following (a) to (j):
(a) mvaE of Enterococcus faecalis;
(b) mvaS of Enterococcus faecalis;
(c) thiL of Lactococcus lactis;
(d) mvaA of Lactococcus lactis;
(e) hmcC of Lactococcus lactis;
(f) a DNA encoding an amino acid sequence of SEQ ID NO: 2;
(g) a DNA encoding an amino acid sequence of SEQ ID NO: 4;
(h) a DNA encoding an amino acid sequence of SEQ ID NO: 12;
(i) a DNA encoding an amino acid sequence of SEQ ID NO: 14; and
(j) a DNA encoding an amino acid sequence of SEQ ID NO: 16.

14. The process according to claim 3, wherein the microorganism not having a mevalonic acid pathway is a microorganism belonging to a genus selected from the group consisting of genus Escherichia, genus Serratia, genus Bacillus, genus Brevibacterium, genus Corynebacterium, genus Microbacterium, genus Pseudomonas and genus Synechocystis.

15. The process according to claim 14, wherein the microorganism belonging to genus Escherichia is Escherichia coli.

16. The process according to any one of claims 1 to 3, wherein the microorganism having the ability to biosynthesize mevalonic acid from acetyl-CoA is a microorganism obtainable by transfecting a DNA participating in biosynthesis of mevalonic acid from acetyl-CoA into a host microorganism using an expression vector.

17. A microorganism having the ability to biosynthesize inevalonic acid from acetyl-CoA and being obtainable by transfecting a DNA participating in biosynthesis of mevalonic acid from acetyl-CoA into a host microorganism.

18. The microorganism according to claim 17, wherein the host microorganism is a microorganism not having the ability to biosynthesize mevalonic acid from acetyl-CoA.

19. The microorganism according to claim 18, wherein the microorganism not having the ability to biosynthesize mevalonic acid from acetyl-CoA is a microorganism not having a mevalonic acid pathway.

20. The microorganism according to any one of claims 17 to 19, wherein the DNA to be transfected is one or more DNA(s) selected from the group consisting of a DNA encoding an enzyme having the activity to produce acetoacetyl-CoA from acetyl-CoA, a DNA encoding an enzyme having the activity to produce HMG-CoA from acetoacetyl-CoA, and a DNA encoding an enzyme having the activity to produce mevalonic acid from HMG-CoA.

21. The microorganism according to claim 20, wherein the DNA encoding an enzyme having the activity to produce acetoacetyl-CoA from acetyl-CoA is a DNA encoding acetyl-CoA acetyltransferase.

22. The microorganism according to claim 20, wherein the DNA encoding an enzyme having the activity to produce HMG-CoA from acetoacetyl-CoA is a DNA encoding HMG-CoA synthase.

23. The microorganism according to claim 20, wherein the DNA encoding an enzyme having the activity to produce mevalonic acid from HMG-CoA is a DNA encoding HMG-CoA reductase.

24. The microorganism according to claim 20, wherein the DNA encoding an enzyme having the activity to produce acetoacetyl-CoA from acetyl-CoA and the DNA encoding an enzyme having the activity to produce mevalonic acid from HMG-CoA are a DNA encoding acetyl-CoA acetyltransferase/HMG-CoA reductase.

25. The microorganism according to any one of claims 17 to 19, wherein the DNA to be transfected is a DNA encoding acetyl-CoA acetyltransferase, a DNA encoding HMG-CoA synthase and a DNA encoding HMG-CoA reductase.

26. The microorganism according to any one of claims 17 to 19, wherein the DNA to be transfected is a DNA encoding acetyl-CoA acetyltransferase/HMG-CoA reductase and a DNA encoding HMG-CoA synthase.

27. The microorganism according to claim 24, wherein the DNA is derived from one or more microorganism(s) selected from the group consisting of bacteria, mycobacteria, archaebacteria and yeast.

28. The microorganism according to claim 24, wherein the DNA is derived from a microorganism belonging to a genus selected from the group consisting of genus Enterococcus, genus Streptococcus, genus Staphylococcus, genus Lactococcus, genus Lactobacillus, genus Streptomyces, genus Methanothermobacter, genus Methanococcus, genus Pyrococcus, genus Thermoplasma and genus Saccharomyces.

29. The microorganism according to any one of claims 17 to 19, wherein the DNA to be transfected is one or more DNA(s) selected from the group consisting of the following (a) to (j):
(a) mvaE of Enterococcus faecalis;
(b) mvaS of Enterococcus faecalis;
(c) thiL of Lactococcus lactis;
(d) mvaA of Lactococcus lactis;
(e) hmcC of Lactococcus lactis;
(f) a DNA encoding an amino acid sequence of SEQ ID NO: 2;
(g) a DNA encoding an amino acid sequence of SEQ ID NO: 4;
(h) a DNA encoding an amino acid sequence of SEQ ID NO: 12;
(i) a DNA encoding an amino acid sequence of SEQ ID NO: 14; and
(j) a DNA encoding an amino acid sequence of SEQ ID NO: 16.

30. The microorganism according to claim 19, wherein the microorganism not having a mevalonic acid pathway is a microorganism belonging to a genus selected from the group consisting of genus Escherichia, genus Serratia, genus Bacillus, genus Brevibacterium, genus Corynebacterium, genus Microbacterium, genus Pseudomonas and genus Synechocystis.

31. The microorganism according to claim 30, wherein the microorganism belonging to genus Escherichia is Escherichia coli.

32. The microorganism according to any one of claims 17 to 19, wherein the microorganism having the ability to biosynthesize mevalonic acid from acetyl-CoA is a microorganism obtainable by transfecting a DNA participating in biosynthesis of mevalonic acid from acetyl-CoA into a host microorganism using an expression vector.


SEQUENCE LISTING (22 sequences)

SEQ ID NO: 1 - DNA, 2412 bp, Enterococcus faecalis
  Inventor Tabata, Kazuhiko; Hashimoto, Shinichi
ttgaaaacagtagttattattgatgcattacgaacaccaattggaaaatataaaggcagcttaagtcaagtaagtgccgtagacttaggaacacatgttacaacacaacttttaaaaagacattccactatttctgaagaaattgatcaagtaatctttggaaatgttttacaagctggaaatggccaaaatcccgcacgacaaatagcaataaacagcggtttatctcatgaaattcccgcaatgacagttaatgaggtctgcggatcaggaatgaaggccgttattttggcgaaacaattgattcaattaggagaagcggaagttttaattgctggcgggattgagaatatgtcccaagcacctaaattacaacgatttaattacgaaacagaaagctatgatgcgcctttttctagtatgatgtacgatgggttaacggatgcctttagtggtcaagcaatgggcttaactgctgaaaatgtggccgaaaagtatcatgtaactagagaagagcaagatcaattttctgtacattcacaattaaaagcagctcaagcacaagcagaagggatattcgctgacgaaatagccccattagaagtatcaggaacgcttgtggagaaagatgaagggattcgccctaattcgagcgttgagaagctaggaacgcttaaaacagtttttaaagaagacggtactgtaacagcagggaatgcatcaaccattaatgatggggcttctgctttgattattgcttcacaagaatatgccgaagcacacggtcttccttatttagctattattcgagacagtgtggaagtcggtattgatccagcctatatgggaatttcgccgattaaagccattcaaaaactgttagcgcgcaatcaacttactacggaagaaattgatctgtatgaaatcaacgaagcatttgcagcaacttcaatcgtggtccaaagagaactggctttaccagaggaaaaggtcaacatttatggtggcggtatttcattaggtcatgcgattggtgccacaggtgctcgtttattaacgagtttaagttatcaattaaatcaaaaagaaaagaaatatggagtggcttctttatgtatcggcggtggcttaggactcgctatgctactagagagacctcagcaaaaaaaaaacagccgattttatcaaatgagtcctgaggaacgcctggcttctcttcttaatgaaggccagatttctgctgatacaaaaaaagaatttgaaaatacggctttatcttcgcagattgccaatcatatgattgaaaatcaaatcagtgaaacagaagtgccgatgggcgttggcttacatttaacagtggacgaaactgattatttggtaccaatggcgacagaagagccctcagtgattgcggctttgagtaatggtgcaaaaatagcacaaggatttaaaacagtgaatcaacaacgtttaatgcgtggacaaatcgttttttacgatgttgcagacgccgagtcattgattgatgaactacaagtaagagaaacggaaatttttcaacaagcagagttaagttatccatctatcgttaaacgcggcggcggcttaagagatttgcaatatcgtgcttttgatgaatcatttgtatctgtcgactttttagtagatgttaaggatgcaatgggggcaaatatcgttaacgctatgttggaaggtgtggccgagttgttccgtgaatggtttgcggagcaaaagattttattcagtattttaagtaattatgccacggagtcggttgttacgatgaaaacggctattccagtttcacgtttaagtaaggggagcaatggccgggaaattgctgaaaaaattgttttagcttcacgctatgcttcattagatccttatcgggcagtcacgcataacaaagggatcatgaatggcattgaagctgtcgttttagctacaggaaatgatacacgcgctgttagcgcttcttgtcatgcttttgcggtgaaggaaggtcgctaccaaggtttgactagttggacgctggatggcgaacaactaattggtgaaatttcagttccgcttgcgttagccacggttggcggtgccacaaaagtcttacctaaatctcaagcagctgctgatttgttagcagtgacggatgcaaaagaactaagtcgagtagtagcggctgttggtttggcacaaaatttagcggcgttacgggccttagtctctgaaggaattcaaaaaggacacatggctctacaagcacgttctttagcgatgacggtcggagctactggtaaagaagttgaggcagtcgctcaacaattaaaacgtcaaaaaacgatgaaccaagaccgagccttggctattttaaatgatttaagaaaacaataa

SEQ ID NO: 2 - protein, 803 aa, Enterococcus faecalis
MKTVVIIDALRTPIGKYKGSLSQVSAVDLGTHVTTQLLKRHSTISEEIDQVIFGNVLQAGNGQNPARQIAINSGLSHEIPAMTVNEVCGSGMKAVILAKQLIQLGEAEVLIAGGIENMSQAPKLQRFNYETESYDAPFSSMMYDGLTDAFSGQAMGLTAENVAEKYHVTREEQDQFSVHSQLKAAQAQAEGIFADEIAPLEVSGTLVEKDEGIRPNSSVEKLGTLKTVFKEDGTVTAGNASTINDGASALIIASQEYAEAHGLPYLAIIRDSVEVGIDPAYMGISPIKAIQKLLARNQLTTEEIDLYEINEAFAATSIVVQRELALPEEKVNIYGGGISLGHAIGATGARLLTSLSYQLNQKEKKYGVASLCIGGGLGLAMLLERPQQKKNSRFYQMSPEERLASLLNEGQISADTKKEFENTALSSQIANHMIENQISETEVPMGVGLHLTVDETDYLVPMATEEPSVIAALSNGAKIAQGFKTVNQQRLMRGQIVFYDVADAESLIDELQVRETEIFQQAELSYPSIVKRGGGLRDLQYRAFDESFVSVDFLVDVKDAMGANIVNAMLEGVAELFREWFAEQKILFSILSNYATESVVTMKTAIPVSRLSKGSNGREIAEKIVLASRYASLDPYRAVTHNKGIMNGIEAVVLATGNDTRAVSASCHAFAVKEGRYQGLTSWTLDGEQLIGEISVPLALATVGGATKVLPKSQAAADLLAVTDAKELSRVVAAVGLAQNLAALRALVSEGIQKGHMALQARSLAMTVGATGKEVEAVAQQLKRQKTMNQDRALAILNDLRKQ

SEQ ID NO: 3 - DNA, 1152 bp, Enterococcus faecalis
  CDS (1)..(1152)
atgacaattgggattgataaaattagtttttttgtgcccccttattatattgatatgacggcactggctgaagccagaaatgtagaccctggaaaatttcatattggtattgggcaagaccaaatggcggtgaacccaatcagccaagatattgtgacatttgcagccaatgccgcagaagcgatcttgaccaaagaagataaagaggccattgatatggtgattgtcgggactgagtccagtatcgatgagtcaaaagcggccgcagttgtcttacatcgtttaatggggattcaacctttcgctcgctctttcgaaatcaaggaagcttgttacggagcaacagcaggcttacagttagctaagaatcacgtagccttacatccagataaaaaagtcttggttgtagcagcagatattgcaaaatatggattaaattctggcggtgagcctacacaaggagctggggcggttgcaatgttagttgctagtgaaccgcgcatcttggctttaaaagaggataatgtgatgctgacgcaagatatctatgacttttggcgtccaacaggccatccgtatcctatggtcgatggtcctttgtcaaacgaaacctacatccaatcttttgcccaagtctgggatgaacataaaaaaagaaccggtcttgattttgcagattatgatgctttagcgttccatattccttacacaaaaatgggcaaaaaagccttattagcaaaaatctccgaccaaactgaagcagaacaggaacgaattttagcccgttatgaagaaagcatcatctatagtcgtcgcgtaggaaacttgtatacgggttcactttatctgggactcatttcccttttagaaaatgcaacgactttaaccgcaggcaatcaaattgggttattcagttatggttctggtgctgtcgctgaatttttcactggtgaattagtagctggttatcaaaatcatttacaaaaagaaactcatttagcactgctagataatcggacagaactttctatcgctgaatatgaagccatgtttgcagaaactttagacacagatattgatcaaacgttagaagatgaattaaaatatagtatttctgctattaataataccgttcgctcttatcgaaactaa

SEQ ID NO: 4 - protein, 383 aa, Enterococcus faecalis
MTIGIDKISFFVPPYYIDMTALAEARNVDPGKFHIGIGQDQMAVNPISQDIVTFAANAAEAILTKEDKEAIDMVIVGTESSIDESKAAAVVLHRLMGIQPFARSFEIKEACYGATAGLQLAKNHVALHPDKKVLVVAADIAKYGLNSGGEPTQGAGAVAMLVASEPRILALKEDNVMLTQDIYDFWRPTGHPYPMVDGPLSNETYIQSFAQVWDEHKKRTGLDFADYDALAFHIPYTKMGKKALLAKISDQTEAEQERILARYEESIIYSRRVGNLYTGSLYLGLISLLENATTLTAGNQIGLFSYGSGAVAEFFTGELVAGYQNHLQKETHLALLDNRTELSIAEYEAMFAETLDTDIDQTLEDELKYSISAINNTVRSYRN

SEQ ID NO: 5 - DNA, 33 bp, Artificial
  primer A
tatatcgatatttagatgaaaacagtagttatt

SEQ ID NO: 6 - DNA, 33 bp, Artificial
  primer B
aatggatccttattgttttcttaaatcatttaa

SEQ ID NO: 7 - DNA, 33 bp, Artificial
  primer C
tatagatctaaggaaggagttaaagaaatgaca

SEQ ID NO: 8 - DNA, 32 bp, Artificial
  primer D
tttggatccttagtttcgataagagcgaacgg

SEQ ID NO: 9 - DNA, 2418 bp, Enterococcus faecalis
  CDS (7)..(2418)
atttagttgaaaacagtagttattattgatgcattacgaacaccaattggaaaatataaaggcagcttaagtcaagtaagtgccgtagacttaggaacacatgttacaacacaacttttaaaaagacattccactatttctgaagaaattgatcaagtaatctttggaaatgttttacaagctggaaatggccaaaatcccgcacgacaaatagcaataaacagcggtttatctcatgaaattcccgcaatgacagttaatgaggtctgcggatcaggaatgaaggccgttattttggcgaaacaattgattcaattaggagaagcggaagttttaattgctggcgggattgagaatatgtcccaagcacctaaattacaacgatttaattacgaaacagaaagctatgatgcgcctttttctagtatgatgtacgatgggttaacggatgcctttagtggtcaagcaatgggcttaactgctgaaaatgtggccgaaaagtatcatgtaactagagaagagcaagatcaattttctgtacattcacaattaaaagcagctcaagcacaagcagaagggatattcgctgacgaaatagccccattagaagtatcaggaacgcttgtggagaaagatgaagggattcgccctaattcgagcgttgagaagctaggaacgcttaaaacagtttttaaagaagacggtactgtaacagcagggaatgcatcaaccattaatgatggggcttctgctttgattattgcttcacaagaatatgccgaagcacacggtcttccttatttagctattattcgagacagtgtggaagtcggtattgatccagcctatatgggaatttcgccgattaaagccattcaaaaactgttagcgcgcaatcaacttactacggaagaaattgatctgtatgaaatcaacgaagcatttgcagcaacttcaatcgtggtccaaagagaactggctttaccagaggaaaaggtcaacatttatggtggcggtatttcattaggtcatgcgattggtgccacaggtgctcgtttattaacgagtttaagttatcaattaaatcaaaaagaaaagaaatatggagtggcttctttatgtatcggcggtggcttaggactcgctatgctactagagagacctcagcaaaaaaaaaacagccgattttatcaaatgagtcctgaggaacgcctggcttctcttcttaatgaaggccagatttctgctgatacaaaaaaagaatttgaaaatacggctttatcttcgcagattgccaatcatatgattgaaaatcaaatcagtgaaacagaagtgccgatgggcgttggcttacatttaacagtggacgaaactgattatttggtaccaatggcgacagaagagccctcagtgattgcggctttgagtaatggtgcaaaaatagcacaaggatttaaaacagtgaatcaacaacgtttaatgcgtggacaaatcgttttttacgatgttgcagacgccgagtcattgattgatgaactacaagtaagagaaacggaaatttttcaacaagcagagttaagttatccatctatcgttaaacgcggcggcggcttaagagatttgcaatatcgtgcttttgatgaatcatttgtatctgtcgactttttagtagatgttaaggatgcaatgggggcaaatatcgttaacgctatgttggaaggtgtggccgagttgttccgtgaatggtttgcggagcaaaagattttattcagtattttaagtaattatgccacggagtcggttgttacgatgaaaacggctattccagtttcacgtttaagtaaggggagcaatggccgggaaattgctgaaaaaattgttttagcttcacgctatgcttcattagatccttatcgggcagtcacgcataacaaagggatcatgaatggcattgaagctgtcgttttagctacaggaaatgatacacgcgctgttagcgcttcttgtcatgcttttgcggtgaaggaaggtcgctaccaaggtttgactagttggacgctggatggcgaacaactaattggtgaaatttcagttccgcttgcgttagccacggttggcggtgccacaaaagtcttacctaaatctcaagcagctgctgatttgttagcagtgacggatgcaaaagaactaagtcgagtagtagcggctgttggtttggcacaaaatttagcggcgttacgggccttagtctctgaaggaattcaaaaaggacacatggctctacaagcacgttctttagcgatgacggtcggagctactggtaaagaagttgaggcagtcgctcaacaattaaaacgtcaaaaaacgatgaaccaagaccgagccttggctattttaaatgatttaagaaaacaataa

SEQ ID NO: 10 - DNA, 1166 bp, Enterococcus faecalis
  CDS (15)..(1166)
aaggagttaaagaaatgacaattgggattgataaaattagtttttttgtgcccccttattatattgatatgacggcactggctgaagccagaaatgtagaccctggaaaatttcatattggtattgggcaagaccaaatggcggtgaacccaatcagccaagatattgtgacatttgcagccaatgccgcagaagcgatcttgaccaaagaagataaagaggccattgatatggtgattgtcgggactgagtccagtatcgatgagtcaaaagcggccgcagttgtcttacatcgtttaatggggattcaacctttcgctcgctctttcgaaatcaaggaagcttgttacggagcaacagcaggcttacagttagctaagaatcacgtagccttacatccagataaaaaagtcttggttgtagcagcagatattgcaaaatatggattaaattctggcggtgagcctacacaaggagctggggcggttgcaatgttagttgctagtgaaccgcgcatcttggctttaaaagaggataatgtgatgctgacgcaagatatctatgacttttggcgtccaacaggccatccgtatcctatggtcgatggtcctttgtcaaacgaaacctacatccaatcttttgcccaagtctgggatgaacataaaaaaagaaccggtcttgattttgcagattatgatgctttagcgttccatattccttacacaaaaatgggcaaaaaagccttattagcaaaaatctccgaccaaactgaagcagaacaggaacgaattttagcccgttatgaagaaagcatcatctatagtcgtcgcgtaggaaacttgtatacgggttcactttatctgggactcatttcccttttagaaaatgcaacgactttaaccgcaggcaatcaaattgggttattcagttatggttctggtgctgtcgctgaatttttcactggtgaattagtagctggttatcaaaatcatttacaaaaagaaactcatttagcactgctagataatcggacagaactttctatcgctgaatatgaagccatgtttgcagaaactttagacacagatattgatcaaacgttagaagatgaattaaaatatagtatttctgctattaataataccgttcgctcttatcgaaactaa

SEQ ID NO: 11 - DNA, 1140 bp, Lactococcus lactis
  CDS (1)..(1140)
gtgaaagaaattgtaatcattgacgccttaagaacaccagttggaagatacgacggtgctcttgctgaatattcagcagcagaacttgggacacatgttgtttcaaacttattgtcaaaacatgaaaatataaaatcagatgttgaacaagttatttttgggaatgttcttcaagctggtaatggtcaaaatatagcacgtcaaattgcgattcatagtggtctatctaatacagtgcccgcttcaacaatcaacgaagtttgtggttcaggaatgaaagcagtgattttagctaaacaacttttacagcttggtgaagcagaagttgtaatcgctggtgggacagaatctatgtctaatgccccaattataaagaatcgaaaaactcaagaagaaacattaagtatgttaaatgatggcttagttgatgccttctcaggaatttctatggggattattggtgaaactattgctgaacaatttgatataagtcgagcagaccaagacgcttttgcgcaaaactctcaagaaaaagcggtcgctgcaagtcaagccggaatttttaaagaagagattgttgcgattggagaagtcggagtcgacgaaacaccacgtccttcttctagtttagaaaaattagccactttaaggacagctttcaaagaaaatggaactgtgacagcaggtaattcttctcctgttaatgatggagcttcagctgtcattcttgcaacgaaagactatgctgaaaaacatgggattccgtacctagcagaactagttgatagtacagaagttggaattgaccctgctattatgggagtttcaccaattgaagctatcaaaaaattaattaatcgttcagagcttgaactttcagatattgacctgtttgaaattaatgaagcatttgcagcatcaagtcttgctgtcaatcgtgaacttggtttaaatgaatcacaagttaacatttatggtggtgcaattgcattaggtcatgctatcggttcaagtggttctaaaattctcacgactttgtcttatgccttaaaacgtgagcaaaaacgctatggaattgctagtctttgtatcggtggaggcctaggtttggctgttcttttaaaaaatcctaacctttaa

SEQ ID NO: 12 - protein, 379 aa, Lactococcus lactis
MKEIVIIDALRTPVGRYDGALAEYSAAELGTHVVSNLLSKHENIKSDVEQVIFGNVLQAGNGQNIARQIAIHSGLSNTVPASTINEVCGSGMKAVILAKQLLQLGEAEVVIAGGTESMSNAPIIKNRKTQEETLSMLNDGLVDAFSGISMGIIGETIAEQFDISRADQDAFAQNSQEKAVAASQAGIFKEEIVAIGEVGVDETPRPSSSLEKLATLRTAFKENGTVTAGNSSPVNDGASAVILATKDYAEKHGIPYLAELVDSTEVGIDPAIMGVSPIEAIKKLINRSELELSDIDLFEINEAFAASSLAVNRELGLNESQVNIYGGAIALGHAIGSSGSKILTTLSYALKREQKRYGIASLCIGGGLGLAVLLKNPNL

SEQ ID NO: 13 - DNA, 1233 bp, Lactococcus lactis
  CDS (1)..(1233)
atgagaaaaaaattttatcaaatgtcgcccaaagagcgattagattcattgaatctgtcagaagatactcaggaagttttgagtgaaatggctttagatacaaatattttaaacaatctaattgaaaatcaaatttccgaatttgaattgccaatgggacttgctcaaaattttgtcattaatggaaaagaatacatcgttccgatggttacagaagaaccatcagttatagctgctgcgagtaatggagcaaaaatagcggaaagttttacggctaaaatagatgaacggttaatgcgtgggcaaattgtattttatgacgtaaagaaaccagaagaaattattaagaaaatatctgaatgcaaaaatgaaatatttgaacaagctaagctttcgtatccttcaattattaagcgtgggggcggtttgagagaaatttcgagcagacttttttcaagtgagaaatttatttcagttgatttcaaagtggatgtcaaagatgcaatgggtgccaatattattaattctatattagaaggtgtagcagaactttttcgtggctggttttctgaggaaaaaattctgtttagcatcctttcaaactatgcaactgaatctttagtaaaagtgagctgtgaaatttcagttgatgctctttcaaaaaaaacaaatggtttagaaattgctcaaaaaattgcagttgcttcacaatactcaaaaattgatccttaccgtgctagcacacacaataaagggattatgaatgggattaatgctgtaattcttgcaacaggaaatgatacgcgagctatttcagcagcaattcatgcttatgcagcaaaagaaggaacatatcaaggtttggctaaatgggaagttcatgccgaaaagttatttggagagttagaaattcctttacctgtggcgaccgttggtggtggggtgaaagtcttacctaaagcacaagcggcaatggaaattttaggaatcacagatgctagagaactggccaaagttatcgccgcagttggtttagcacaaaatttagcagctttgcgcgcacttgtttcagaaggaattcaacaaggacatatgagcttacaggctcgctctttggcattatccgtgggtgcaaaagcagatgagatagcagtcataagtcagcaacttcggcaggaaaaagtgatgaatcaagaagtagctcgccgtttattaaatagtctcagaaactaa

SEQ ID NO: 14 - protein, 410 aa, Lactococcus lactis
MRKKFYQMSPKERLDSLNLSEDTQEVLSEMALDTNILNNLIENQISEFELPMGLAQNFVINGKEYIVPMVTEEPSVIAAASNGAKIAESFTAKIDERLMRGQIVFYDVKKPEEIIKKISECKNEIFEQAKLSYPSIIKRGGGLREISSRLFSSEKFISVDFKVDVKDAMGANIINSILEGVAELFRGWFSEEKILFSILSNYATESLVKVSCEISVDALSKKTNGLEIAQKIAVASQYSKIDPYRASTHNKGIMNGINAVILATGNDTRAISAAIHAYAAKEGTYQGLAKWEVHAEKLFGELEIPLPVATVGGGVKVLPKAQAAMEILGITDARELAKVIAAVGLAQNLAALRALVSEGIQQGHMSLQARSLALSVGAKADEIAVISQQLRQEKVMNQEVARRLLNSLRN

SEQ ID NO: 15 - DNA, 1155 bp, Lactococcus lactis
  CDS (1)..(1155)
atgaaagtcggtatagataaacttggattttttgtcccaaatacttttgttgatatgagggatttagcaaatgctcgtaatgttgaccctgcaaaattccaaattggtattggtcaagatgaaatggcagtcaatcctgctactcaagatattattacttttgctgctaacgcggcagcaagtattttgactgaggaagataaaaaagccattgacatgatcatcgttggtactgaatcaagtcttgatgaatcaaaggcctcagccgttgttgttcacgatttgcttggtattcaaccttttgctcgaagcattgaaatgaaagaggcttgttatgcaactactgctggccttactcttgctcgtgaccacgttcttttgaatcctgatactaaagttttagttattgcttctgatattgccaaatatgggctaaatactggtggagaacctactcagggcgctggaagtgttgcaatgcttattacggccgatcctaaaattttggccttaaataatgataacgttgctttaactcaagacatctatgatttttggcgtccttttggtcaggcttatccttctgttgatggtaagttttcaaacgaaacatatatcgatgcttttgctaaaatctggaaagagtatagccatagaacaaatttaaagtttgaagactttgcagctatcgcctttcataccccttatactaaaatgggtaaaaaagctttattgccaatgcttgaatctgaaaaaccagccaatgctgaagaattaatggaacaatttgaacatggtatcgtttataatcgtcgtgtgggtaatctgtataccggttctctctatttgagtttgatttcacttttggaaaactctgataaattgagtgctggtcagcgtattggactttttagttatggttctggtactgttgctgagtttttctctggtgagcttgttgaaggttatgaaaatcatttacttaaaattgaacatcaatcaatgttggatgcaagaacccgtctatcaattcctgaatacgaaaacatgtttaatcaatatttagatttgaatcataatatttctttcaatgatgagacagattactccgtttcagaagttgttgataatcaccgtaagtataaaaaaagataa

SEQ ID NO: 16 - protein, 384 aa, Lactococcus lactis
MKVGIDKLGFFVPNTFVDMRDLANARNVDPAKFQIGIGQDEMAVNPATQDIITFAANAAASILTEEDKKAIDMIIVGTESSLDESKASAVVVHDLLGIQPFARSIEMKEACYATTAGLTLARDHVLLNPDTKVLVIASDIAKYGLNTGGEPTQGAGSVAMLITADPKILALNNDNVALTQDIYDFWRPFGQAYPSVDGKFSNETYIDAFAKIWKEYSHRTNLKFEDFAAIAFHTPYTKMGKKALLPMLESEKPANAEELMEQFEHGIVYNRRVGNLYTGSLYLSLISLLENSDKLSAGQRIGLFSYGSGTVAEFFSGELVEGYENHLLKIEHQSMLDARTRLSIPEYENMFNQYLDLNHNISFNDETDYSVSEVVDNHRKYKKR

SEQ ID NO: 17 - DNA, 30 bp, Artificial
  primer E
atgatcgatggaggttcattcatgaaagaa

SEQ ID NO: 18 - DNA, 30 bp, Artificial
  primer F
agtggatccttagtttctgagactatttaa

SEQ ID NO: 19 - DNA, 26 bp, Artificial
  primer G
tttagatctaataagtgaggaaattt

SEQ ID NO: 20 - DNA, 30 bp, Artificial
  primer H
agtggatccttatctttttttatacttacg

SEQ ID NO: 21 - DNA, 2464 bp, Lactococcus lactis
  CDS (13)..(1152)
  CDS (1232)..(2464)
ggaggttcattcgtgaaagaaattgtaatcattgacgccttaagaacaccagttggaagatacgacggtgctcttgctgaatattcagcagcagaacttgggacacatgttgtttcaaacttattgtcaaaacatgaaaatataaaatcagatgttgaacaagttatttttgggaatgttcttcaagctggtaatggtcaaaatatagcacgtcaaattgcgattcatagtggtctatctaatacagtgcccgcttcaacaatcaacgaagtttgtggttcaggaatgaaagcagtgattttagctaaacaacttttacagcttggtgaagcagaagttgtaatcgctggtgggacagaatctatgtctaatgccccaattataaagaatcgaaaaactcaagaagaaacattaagtatgttaaatgatggcttagttgatgccttctcaggaatttctatggggattattggtgaaactattgctgaacaatttgatataagtcgagcagaccaagacgcttttgcgcaaaactctcaagaaaaagcggtcgctgcaagtcaagccggaatttttaaagaagagattgttgcgattggagaagtcggagtcgacgaaacaccacgtccttcttctagtttagaaaaattagccactttaaggacagctttcaaagaaaatggaactgtgacagcaggtaattcttctcctgttaatgatggagcttcagctgtcattcttgcaacgaaagactatgctgaaaaacatgggattccgtacctagcagaactagttgatagtacagaagttggaattgaccctgctattatgggagtttcaccaattgaagctatcaaaaaattaattaatcgttcagagcttgaactttcagatattgacctgtttgaaattaatgaagcatttgcagcatcaagtcttgctgtcaatcgtgaacttggtttaaatgaatcacaagttaacatttatggtggtgcaattgcattaggtcatgctatcggttcaagtggttctaaaattctcacgactttgtcttatgccttaaaacgtgagcaaaaacgctatggaattgctagtctttgtatcggtggaggcctaggtttggctgttcttttaaaaaatcctaacctttaaaaattattgtggtttaaacaagtaatttttaagaaattaatggaggaattgcccagcttatctgggcatttttgaaaatatgagaaaaaaattttatcaaatgtcgcccaaagagcgattagattcattgaatctgtcagaagatactcaggaagttttgagtgaaatggctttagatacaaatattttaaacaatctaattgaaaatcaaatttccgaatttgaattgccaatgggacttgctcaaaattttgtcattaatggaaaagaatacatcgttccgatggttacagaagaaccatcagttatagctgctgcgagtaatggagcaaaaatagcggaaagttttacggctaaaatagatgaacggttaatgcgtgggcaaattgtattttatgacgtaaagaaaccagaagaaattattaagaaaatatctgaatgcaaaaatgaaatatttgaacaagctaagctttcgtatccttcaattattaagcgtgggggcggtttgagagaaatttcgagcagacttttttcaagtgagaaatttatttcagttgatttcaaagtggatgtcaaagatgcaatgggtgccaatattattaattctatattagaaggtgtagcagaactttttcgtggctggttttctgaggaaaaaattctgtttagcatcctttcaaactatgcaactgaatctttagtaaaagtgagctgtgaaatttcagttgatgctctttcaaaaaaaacaaatggtttagaaattgctcaaaaaattgcagttgcttcacaatactcaaaaattgatccttaccgtgctagcacacacaataaagggattatgaatgggattaatgctgtaattcttgcaacaggaaatgatacgcgagctatttcagcagcaattcatgcttatgcagcaaaagaaggaacatatcaaggtttggctaaatgggaagttcatgccgaaaagttatttggagagttagaaattcctttacctgtggcgaccgttggtggtggggtgaaagtcttacctaaagcacaagcggcaatggaaattttaggaatcacagatgctagagaactggccaaagttatcgccgcagttggtttagcacaaaatttagcagctttgcgcgcacttgtttcagaaggaattcaacaaggacatatgagcttacaggctcgctctttggcattatccgtgggtgcaaaagcagatgagatagcagtcataagtcagcaacttcggcaggaaaaagtgatgaatcaagaagtagctcgccgtttattaaatagtctcagaaactaa

SEQ ID NO: 22 - DNA, 1179 bp, Lactococcus lactis
  CDS (25)..(1179)
aataagtgaggaaatttttaaaatatgaaagtcggtatagataaacttggattttttgtcccaaatacttttgttgatatgagggatttagcaaatgctcgtaatgttgaccctgcaaaattccaaattggtattggtcaagatgaaatggcagtcaatcctgctactcaagatattattacttttgctgctaacgcggcagcaagtattttgactgaggaagataaaaaagccattgacatgatcatcgttggtactgaatcaagtcttgatgaatcaaaggcctcagccgttgttgttcacgatttgcttggtattcaaccttttgctcgaagcattgaaatgaaagaggcttgttatgcaactactgctggccttactcttgctcgtgaccacgttcttttgaatcctgatactaaagttttagttattgcttctgatattgccaaatatgggctaaatactggtggagaacctactcagggcgctggaagtgttgcaatgcttattacggccgatcctaaaattttggccttaaataatgataacgttgctttaactcaagacatctatgatttttggcgtccttttggtcaggcttatccttctgttgatggtaagttttcaaacgaaacatatatcgatgcttttgctaaaatctggaaagagtatagccatagaacaaatttaaagtttgaagactttgcagctatcgcctttcataccccttatactaaaatgggtaaaaaagctttattgccaatgcttgaatctgaaaaaccagccaatgctgaagaattaatggaacaatttgaacatggtatcgtttataatcgtcgtgtgggtaatctgtataccggttctctctatttgagtttgatttcacttttggaaaactctgataaattgagtgctggtcagcgtattggactttttagttatggttctggtactgttgctgagtttttctctggtgagcttgttgaaggttatgaaaatcatttacttaaaattgaacatcaatcaatgttggatgcaagaacccgtctatcaattcctgaatacgaaaacatgtttaatcaatatttagatttgaatcataatatttctttcaatgatgagacagattactccgtttcagaagttgttgataatcaccgtaagtataaaaaaagataa